(19) United States
(12) Patent Application Publication
(10) Pub. No.: US 2007/0135437 A1
(43) Pub. Date: Jun. 14, 2007

(54) Modulation of neurodegenerative diseases

(75) Inventors: Daniel E. Benjamin, Englishtown, NJ (US); Robert N. Henrie II, Pennington, NJ (US); Monica A. Errico, Annandale, NJ (US); Brian T. Rex, Lake Hiawatha, NJ (US); Justin D. Bracken, Philadelphia, PA (US); Sean Scott, San Francisco, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ALSGEN, INC., San Francisco, CA (US)

(21) Appl. No.: 11/605,170

(22) Filed: Nov. 28, 2006

Related U.S. Application Data
(63) Continuation-in-part of application No. 11/365,462, filed on Mar. 1, 2006.
(60) Provisional application No. 60/658,505, filed on Mar. 4, 2005.

(51) Int. Cl. A61K 31/53; A61K 31/505; A61K 31/513

(52) U.S. Cl. 514/241; 514/269; 514/275

(57) ABSTRACT

Methods and compositions are disclosed for selectively decreasing protein levels in a central nervous system, meningial, immune system, blood, or muscle cell by administrating a pharmacological agent. In particular, methods and compositions that interfere with SOD-1 protein synthesis or stability, and decrease cellular levels of the protein are disclosed.

### RELATED APPLICATIONS

This application is a continuation-in-part of U.S. application Ser. No. 11/365,462, filed Mar. 1, 2005, which claims benefit of priority to U.S. Provisional Application No. 60/658,505, filed Mar. 4, 2005, the entire disclosure of which is incorporated herein by reference in their entirety.

### BACKGROUND OF THE INVENTION

Amyotrophic lateral sclerosis (ALS) is the most commonly diagnosed progressive motor neuron disease. The disease is characterized by degeneration of motor neurons in the cortex, brainstem and spinal cord (Principles of Internal Medicine, 1991 McGraw-Hill, Inc., New York; Tandan et al. (1985) Ann. Neurol, 18:271-280, 419-431). The cause of the disease is unknown and ALS may only be diagnosed when the patient begins to experience asymmetric limb weakness and fatigue, localized fasciculation in the upper limbs and/or spasticity in the legs which typifies onset. There is a genetic component to at least some incidences of ALS.

In almost all instances, sporadic ALS and autosomal dominant familial ALS (FALS) are clinically similar (Mulder et al. (1986) Neurology, 36:511-517). It has been shown that in some but not all FALS pedigrees the disease is linked to a genetic defect on chromosome 21q (Siddique et al., (1991) New Engl. J. Med., 324:1381-1384).

In particular, mutations in the SOD-1 gene which is localized on chromosome 21q, appear to be associated with the familial form of ALS. The deleterious effects of various mutations on SOD-1 are most likely mediated through a gain of toxic function rather than a loss of SOD-1 activity (Al-Chalabi and Leigh, (2000) Curr. Opin. Neurol., 13, 397-405; Alisky et al. (2000) Hum. Gene Ther., 11, 2315-2329). While the toxicity is unclear, there exists evidence to suggest that elimination of the protein itself will ameliorate the toxicity.

A need exists to develop therapies that can alter the course of neurodegenerative diseases or prolong the survival time of patients with such diseases. In particular, a need exists to reduce the SOD-1 protein produced in the brain and spinal cord of ALS patients.

### SUMMARY OF THE INVENTION

Methods and compositions are disclosed for interfering with protein synthesis in the brain, spinal cord, meningial and muscle cells by administrating a pharmacological agent.

Accordingly, in one aspect, the invention pertains to a method for reducing the levels of the SOD-1 protein in a cell comprising administering a pharmacological agent to the cell, such that the agent inhibits expression of the SOD-1 protein. The cell can be a neural cell, or any cell in the spinal cord, the meningial tissue, or a muscle cell, for example in a subject with ALS (e.g., familial ALS). The SOD protein can be the SOD-1 protein. Examples of cells include, but are not limited to, neurons, interneurons, glial cells, microglia cells, muscle cells, cells involved in the immune response, and the like.

In another aspect, the invention pertains to a method for preventing, ameliorating or treating the symptoms or progression of ALS in a subject by administering a therapeutically effective amount of a pharmacological agent to the subject, wherein the agent interacts with the nucleus of the cell and inhibits transcription of a gene encoding a SOD-1 protein, prevents the synthesis of the protein, or accelerates the disposition or destruction of the protein. The ameliorating of symptoms can be monitored by measuring the survival prolongation of the subject, for example by monitoring a neurological score of the subject. Alternatively, the amelioration can be determined by monitoring the expression levels of the SOD-1 protein or the levels of a nucleic acid molecule that encodes SOD-1 protein.

In another aspect, the invention discloses a method for reducing the levels of an SOD protein in a cell comprising, administering a pharmacological agent to the cell, such that the agent decreases levels of the SOD protein, wherein the agent is a compound of Formula I 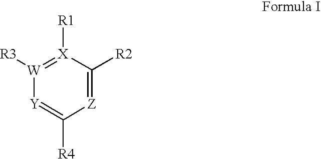
wherein W, X, Y, and Z are independently selected from C, N, O, S, with at least one being non-carbon. R1 through R4 are independently selected from: H, halogen, cyano, SCN, alkyl, haloalkyl, hydroxyalkyl, alkoxyalkyl, aryloxyalkyl, carboxyalkyl, carboalkoxyalkyl, arylalkyl, heteroarylalkyl, cycloalkyl, mercaptoalkyl, alkylthioalkyl, alkylsulfonylalkyl, alkylsulfoxylalkyl, acyl (eg. acetyl), alkenyl, alkynyl, arylalkenyl, arylalkynyl, aryloxyalkenyl, aryloxyalkynyl, arylthioalkenyl, arylthioalkynyl, heteroarylalkenyl, heteroarylalkynyl, heteroaryloxyalkenyl, heteroaryloxyalkynyl, heteroarylthioalkenyl, heteroarylthioalkynyl, aryl, heteroaryl, aroyl (eg. benzoyl), heteroaroyl, and saturated heterocyclyl (eg. morpholino, thiamorpholino, piperazinyl, piperidinyl, pyrrolidinyl, tetrahydrofuranyl), nitro, amino, alkylamino, dialkylamino, arylamino, heteroarylamino, arylalkylamino, heteroarylalkylamino, acylamino, aroylamino, heteroaroylamino, arylguanidino, CONRaryl, CONRheteroaryl, CO₂H, CO₂R, hydroxy or double-bonded oxygen (to satisfy valence), alkoxy, aryloxy, heteroaryloxy, haloalkoxy, carboxyalkoxy, carboalkoxyalkoxy, aryloxy, heteraryloxy, alkenyloxy, alkynyloxy, OCH₂CO₂R, OCH₂CONR₂, mercapto, alkylthio, alkylsulfonyl, alkylsulfoxyl, arylthio, heteroarylthio, arylalkylthio, heteroarylalkylthio, alkenylthio, alkynylthio, SCH₂CO₂R, and SCH₂CONR₂, where R═H, alkyl, haloalkyl, or alkoxyalkyl. R1, R2, R3, and R4 also may include ring fusions with adjacent positions, giving for example benzo (CH═CH—CH═CH) or saturated groups [eg, (CH₂)n, where n=3-5]. Aryl and heteroaryl groups, either as substituents, parts of other substituents (eg. arylalkyl, heteroarylalkyl), or fused groups, may be optionally substituted with one or more of hydroxy, alkyl, halogen, haloalkyl, alkoxy, haloalkoxy, amino, alkylamino, dialkylamino, acylamino, aroylamino, mercapto, alkylthio, alkylsulfonyl, alkylsulfoxyl, cyano, CO2R, nitro, acyl (eg. acetyl), aroyl (eg. benzoyl).

The cell can be selected from the group consisting of a brain cell, a spinal cord cell, a cell in a meningial tissue, a neuronal cell, and/or a muscle cell. In some embodiments, the cell can be obtained from a subject diagnosed with ALS. The cell can be a neural cell, or any cell in the spinal cord, the meningial tissue, or a muscle cell, for example in a subject with ALS (e.g., familial ALS). The SOD protein can be the SOD-1 protein. Examples of cells include, but are not limited to, neurons, intemeurons, glial cells, microglia cells, muscle cells, cells involved in the immune response, and the like.

The pharmacological agent can be, for example, a pyrimidines, pyridines, 1,3,5-triazines, flavonoids, quinazolines, 1,2,4-triazines, and heterocyclic amides, or combinations thereof. Exemplary compounds are listed in Tables 1 through 5, and corresponding core structures can be found in FIG. 2. The percent reduction of the SOD protein with the pharmacological agents of the invention can be found in Tables 1B, 2-5. In preferred embodiments, the pharmacological agent can reduce the SOD protein by at least about 30%, about 40%, about 50%, about 60%, about 70%, about 75%, about 80%, about 85%, about 90%, about 94%, about 95%, about 96%, about 97%, about 98%, or about 99%. Reduction of SOD protein is referred to as “biological activity.” A sample technique for measuring reduction in SOD protein is described in the Examples Section.

In some embodiments, the pharmacological agent can be a pyrimidine of Formula I, with W,X═C; and Y,Z=N. Exemplary pyrimidines of the present invention are listed in Table 1. In some exemplary embodiments, R1 can be a haloaryl. For example, R1 can be 4-C₆H₄—Cl, R4 can be SH or NH2, R2 can be NH₂ and R3 can be C₂H₅.

The invention discloses, the use of a compound for reducing the production of an SOD protein in a cell, wherein the compound is of formula 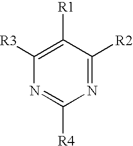
wherein R1 is methoxy, C₆H₄-2-Cl, or C₆H₄-4-OC₃H₇, R2 is NMe₂, NH2 or CF₃, R3 is H or C₂H₅, and R4 is pyrid₂yl-4-OC₃H₇, SH, CF₃, or NH₂. Exemplary compounds are described in Table 1A, as compounds 13, 14, 15, 16, 17 and 36. The SOD protein can be SOD-1.

In other embodiments, the pharmacological agent can be a 1,3,5-triazine of Formula I, with W═C; and X,Y,Z=N. Exemplary triazines of the present invention are listed in Table 2. In some exemplary embodiments, R can be a haloalkyl, such as CHCl₂. R3 can be an alkylthio. R4 can be an aryl.

In other embodiments, the pharmacological agent can be a quinazoline of Formula I, wherein W,Y,Z=C; X═N; and R3 is a benzo fused ring. Exemplary quinazolines of the present invention are listed in Table 4. In some exemplary embodiments, R4 is a saturated heterocycle. R4 can be, for example, 4-methylpiperazin-1-yl. R2 can be CF₃.

In other embodiments, the pharmacological agent can be a heterocyclic amide of Formula I, with W,X,Z=C; Y═N and R4=NRCOaryl. Exemplary heterocyclic amides of the present invention are listed in Table 5.

In other embodiments, the pharmacological agent can be a flavonoid of Formula I, with W,Y,Z=C; X═O and R3 is a benzo fused ring. Exemplary flavonoids of the present invention are listed in Table 3. In some exemplary embodiments, R4 can be a double bonded oxygen. R2 can be H or aryl.

In another aspect, the invention discloses a method for preventing the development of symptoms, or ameliorating the symptoms or progression of amyotrophic lateral sclerosis (ALS) in a subject comprising, administering a prophylactically or therapeutically effective amount of a pharmacological agent to the subject, wherein the agent decreases levels of the SOD-1 protein. In another aspect, the invention discloses a method for preventing the development of symptoms, or ameliorating the symptoms or progression of amyotrophic lateral sclerosis (ALS) in a subject with a mutation in the SOD1 gene, comprising, administering a prophylactically or therapeutically effective amount of a pharmacological agent to the subject, wherein the agent decreases levels of the SOD-1 protein. The pharmacological agents can be particularly useful in treating familial ALS (FALS). The expression and accumulation of mutant SOD-1 is a widely accepted pathophysiological mechanism underlying familial ALS, and might also play a role in the sporadic form of the disease. The pharmacological agent can be, for example, pyrimidines, pyridines, 1,3,5-triazines, flavonoids, quinazolines, 1,2,4-triazines, and heterocyclic amides, or combinations thereof Exemplary compounds are listed in Tables 1 through 5, and core structures can be found in FIG. 2.

The method can further include monitoring the amelioration of ALS by monitoring survival prolongation of the subject. The step of monitoring the amelioration of ALS can include monitoring a neurological score of the subject, monitoring expression levels of the SOD-1 protein, monitoring the clinical measures MMT, ALSFRS/ALSFRS-R, or the Appel Scale, and/or monitoring the number of motor units via the MUNE technique.

### BRIEF DESCRIPTION OF DRAWINGS

FIG. 1 is a graph showing the reduction of SOD-1 protein expression by pyrimethamine.

FIG. 2 is a schematic showing the core structures of the pyrimidines, triazines, flavonoids, quinazolines, and heterocyclic amides of the invention.

FIG. 3 is a bar graph showing reduced expression of mRNA for alpha synuclein in HeLa cells following treatment with pyrimethamine and norethindrone.

FIG. 4 is a bar graph showing the reduction of SOD-1 protein expression in male and female SOD-93A mice with chronic pyrimethamine treatment (TX).

FIG. 5 is a bar graph showing the decrease in expression of alpha synuclein in mouse lymphocytes with chronic pyrimethamine treatment.

FIG. 6 is a bar graph showing the decreased expression of spinal SOD-1 in SOD-93A mice following oral administration of pyrimethamine.

FIG. 7 is a bar graph showing a decrease in lymphocyte SOD-1 levels in a familial SOD-1 patient following 30 days of oral administration of pyrimethamine.

### DETAILED DESCRIPTION

The practice of the present invention employs, unless otherwise indicated, conventional methods of microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tqjessen, ed.); Fundamental Virology, 2nd Edition, Vol. I & II (B. N. Fields and D. M Knipe, eds.)).

So that the invention is more clearly understood, the following terms are defined:

The term “neurodegenerative disorder” or “neurodegenerative disease” are used interchangeably herein and refer to an impairment or absence of a normal neurological function, or presence of an abnormal neurological function in a subject, or group of subjects. For example, neurological disorders can be the result of disease, injury, and/or aging. As used herein, neurodegenerative disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, Amyotrophic Lateral Sclerosis (ALS), multiple sclerosis, Huntington's disease, Parkinson's disease, Alzheimer's disease, prion associated disease (CJD), spinal muscular atrophy, spinal cerebellar ataxia, and spinal cord injury.

The terms “pharmacological agent” as used herein, is intended to refer to the compound or compounds that decrease SOD-1 protein levels in a neural spinal cord, meningial, or muscle cell. In particular, the pharmacological agent decreases the cellular content of an SOD protein, e.g., SOD-1 protein. Preferably, the pharmacological agent is pyrimethamine and analogs thereof.

The terms “modulate” or “modulating” or “modulated” are used interchangeable herein also refer to a change in SOD-1 activity, or the expression, i.e., an increase or decrease in SOD-1 activity, or expression, such that the modulation produces a therapeutic effect in a subject, or group of subjects. A therapeutic effect is one that results in an amelioration in the symptoms, or progression of ALS. The change in activity can be measured by quantitative or qualitative measurements of the SOD-1 protein level for example by Western blot analysis. The quantitative assay can be used to measure downregulation or upregulation of SOD-1 protein levels in the presence of a pharmacological agent, such as pyrimethamine and analogs thereof. A suitable pharmacological agent can be one that down-regulates SOD-1 expression by about 5 percent to about 50 percent or more compared with a control. The change in expression can also be measured by quantitative or qualitative measurements of the nucleic acid level associated with SOD-1, for example by measuring the expression level of RNA or DNA.

The effect of SOD-1 modulation on a subject, or group of subjects, can also be investigated by examining the survival of the subject, or group of subjects. For example, by measuring the change in the survival, or the prolongation of survival in one or more animal models for a neurodegenerative disease, e.g., ALS. The change in the survival can be due to the administration of pharmacological agent such as pyrimethamine or functional analog that is administered to an ALS murine model. The effect of the pharmacological agent can be determined based on the increase in days of survival of a test group of ALS mice compared with a control group of ALS mice that have been given a control agent, or no agent. In one embodiment, the pharmacological agent or functional analog thereof increases the percentage effect on survival of the subject, or a population of subjects (e.g., a male population, or a female population) by at least 2% to about 100%. Preferably the percentage effect on survival of the subject, or a population of subjects, is by at least 5% to about 50%, by at least 10% to about 25%. Even more preferably, the percentage effect on survival of the subject, or a population of subjects, is by at least 3%, 4%, 5%, 6%, 7%, 8%, 9%, 10%, 12%, 14%, 16%, 18%, 20%, 22%, 24%, 26% 28%, 30%, 32%,34%, 36%, 38%, 40%, 42%, 44%, 46%, 48% and 50%. The effect of SOD-1 modulation may also determined by examining the neurological score of a subject, or group of subjects for example, by assessing the improvement in muscular movement, or by examining the alleviation or amelioration of the disease symptoms. In a preferred embodiment, the neurological score of a subject, or group of subjects is significantly different from that of the untreated control subjects, with a level of significance between p<0.05 and p<0.0001, as determined using standard statistical analysis procedures.

The terms may also be used to refer to a change in the nuclear receptor upon interaction with a pharmacological agent, i.e., a change in nuclear receptor activity, structure, or the expression of a nuclear receptor, or a subunit of the nuclear receptor, i.e., an increase or decrease in nuclear receptor activity, or expression, such that the modulation produces a therapeutic effect in a subject, or group of subjects.

The term “inhibit” or “inhibiting” as used herein refers to a measurable reduction of expression of a target gene or a target protein, e.g., SOD-1. The term also refers to a measurable reduction in the activity of a target protein. Preferably a reduction in expression is at least about 10%. More preferably the reduction of expression is about 20%, 30%, 40%, 50%, 60%, and 80%.

The phrase “a disorder associated with SOD activity” or “a disease associated with SOD activity” as used herein refers to any disease state associated with the expression of SOD protein (e.g., SOD-1, SOD-2, SOD-3, and the like). In particular, this phrase refers to the gain of toxic function associated with SOD protein production. The SOD protein can be a wild type SOD protein or a mutant SOD protein and can be derived from a wild type SOD gene or an SOD gene with at least one mutation.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.). Unless otherwise specified the term alkyl includes both “unsubstituted alkyls” and “substituted alkyls,” the latter of which refers to alkyl groups having substituents replacing one or more hydrogens on one or more carbons of the hydrocarbon backbone.

The term “alkoxy group” as used herein means an alkyl group having an oxygen atom attached thereto. Representative alkoxy groups include groups having 1-10 carbon atoms, preferably 1-6 carbon atoms, e.g., methoxy, ethoxy, propoxy, tert-butoxy, and the like. Examples of alkoxy groups include methoxy, ethoxy, propoxy, iso-propoxy, butoxy, pentoxy.

The term “aromatic group” or “aryl group” includes unsaturated and aromatic cyclic hydrocarbons as well as unsaturated and aromatic heterocycles containing one or more rings. Aryl or aromatic groups may also be fused or bridged with alicyclic or heterocyclic rings that are not aromatic so as to form a polycycle (e.g., tetralin), or rings that are aromatic.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). An “alkoxyphenyl” group (or “alkyloxyphenyl” group) is a phenyl group substituted with an alkoxy group (e.g., p-methoxyphenyl). An “arylalkoxy” group is an alkoxy group substituted with a phenyl group (e.g., benzyloxy), An “aryloxyalkyl” group is an alkyl group substituted with an oxyaryl group (e.g., phenylmethyl ether (i.e., phenoxymethyl)), An “aryloxyphenyl” group is an phenyl group substituted with a phenoxy group (e.g., biphenyl ether (i.e., phenoxyphenyl)), A “phenoxy” group is an oxygen atom attached via a phenyl group.

The term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups.

A “heteroarylalkyl” group is an alkyl group in which one of the hydrogens has been replaced with a heteroaryl substituent, eg. 4-pyridylmethyl, 2-furylethyl, etc.

A “heteroarylalkoxy” group is an alkoxy group in which one of the hydrogens has been replaced with a heteroaryl substituent, eg. 4-pyridylmethoxy, 2-furylmethoxy, etc.

I. Neurodegenerative Diseases

In one aspect, the invention pertains to altering the expression of an SOD protein in a cell by administering a pharmacological agent, e.g., a protein modulating pharmacological agent. The cell can be a neural cell associated in a neurodegenerative disease that involves an SOD protein, such as amyotrophic lateral sclerosis (ALS).

Amyotrophic Lateral Sclerosis (ALS), also called Lou Gehrig's disease, is a fatal neurodegenerative disease affecting motor neurons of the cortex, brain stem and spinal cord. (Hirano, (1996) Neurology, 47(4 Suppl. 2): S63-6). Onset of ALS generally occurs in the fourth or fifth decade of life (median age of onset is 57) and is fatal within two to five years after diagnosis (Williams, et al. (1991) Mayo Clin. Proc., 66: 54-82). ALS affects approximately 30,000 Americans with nearly 8,000 deaths reported in the US each year. ALS patients progressively lose all motor function—unable to walk, speak, or breathe on their own.

The cardinal feature of ALS is the loss of spinal motor neurons, which causes the muscles under their control to weaken and waste away leading to paralysis. ALS has both familial (5-10%) and sporadic forms and the familial forms have now been linked to several distinct genetic loci (Deng, et al. (1995) Hum. Mol. Genet., 4: 1113-16; Siddique, et al. (1995) Clin. Neurosci., 3: 338-47; Siddique, et al., (1997) J. Neural Transm. Suppl., 49: 219-33; Ben Hamnida, et al. (1990) Brain, 113: 347-63; Yang, et al. (2001) Nat. Genet. 29: 160-65; Hadano, et al. (2001) Nat. Genet. 29: 166-73). About 15-20% of familial cases are due to mutations in the gene encoding Cu/Zn superoxide dismutase 1 (SOD1) (Siddique, et al. (1991) N. Engl. J. Med., 324: 1381-84; Rosen, et al. (1993) Nature, 362: 59-62).

Although the etiology of the disease is unknown, one theory is that neuronal cell death in ALS is the result of over-excitement of neuronal cells due to excess extracellular glutamate. Glutamate is a neurotransmitter that is released by glutaminergic neurons, and is taken up into glial cells where it is converted into glutamine by the enzyme glutamine synthetase, glutamine then re-enters the neurons and is hydrolyzed by glutaminase to form glutamate, thus replenishing the neurotransmitter pool. In a normal spinal cord and brain stem, the level of extracellular glutamate is kept at low micromolar levels in the extracellular fluid because glial cells, which function in part to support neurons, use the excitatory amino acid transporter type 2 (EAAT2) protein to absorb glutamate immediately. A deficiency in the normal EAAT2 protein in patients with ALS, was identified as being important in the pathology of the disease (See e.g., Meyer et al. (1998) J. Neurol. Neurosurg. Psychiatry, 65: 594-596; Aoki et al. (1998) Ann. Neurol. 43: 645-653; Bristol et al. (1996) Ann Neurol. 39: 676-679). One explanation for the reduced levels of EAAT2 is that EAAT2 is spliced aberrantly (Lin et al. (1998) Neuron, 20: 589-602). The aberrant splicing produces a splice variant with a deletion of 45 to 107 amino acids located in the C-terminal region of the EAAT2 protein (Meyer et al. (1998) Neureosci Lett. 241: 68-70). Due to the lack of, or defectiveness of EAAT2, extracellular glutamate accumulates, causing neurons to fire continuously. The accumulation of glutamate has a toxic effect on neuronal cells because continual firing of the neurons leads to early cell death.

Although a great deal is known about the pathology of ALS, little is known about the pathogenesis of the sporadic form and about the causative properties of mutant SOD protein in familial ALS (Bruijn, et al. (1996) Neuropathol. Appl. Neurobiol., 22: 373-87; Bruijn, et al. (1998) Science 281: 1851-54). Many models have been speculated, including glutamate toxicity, hypoxia, oxidative stress, protein aggregates, neurofilament and mitochondrial dysfunction Cleveland, et al. (1995) Nature 378: 342-43; Cleveland, et al. Neurology, 47(4 Suppl. 2): S54-61, discussion S61-2(1996); Cleveland, (1999) Neuron, 24: 515-20; Cleveland, et al. (2001) Nat. Rev. Neurosci., 2: 806-19; Couillard-Despres, et al. (1998) Proc. Natl. Acad. Sci. USA, 95: 9626-30; Mitsumoto, (1997) Ann. Pharmacother., 31: 779-81; Skene, et al. (2001) Nat. Genet. 28: 107-8; Williamson, et al. (2000) Science, 288: 399).

Presently, there is no cure for ALS, nor is there a therapy that has been proven effective to prevent or reverse the course of the disease. Several drugs have recently been approved by the Food and Drug Administration (FDA). To date, attempts to treat ALS have involved treating neuronal degeneration with long-chain fatty alcohols which have cytoprotective effects (See U.S. Pat. No. 5,135,956); or with a salt of pyruvic acid (See U.S. Pat. No. 5,395,822); and using a glutamine synthetase to block the glutamate cascade (See U.S. Pat. No. 5,906,976). For example, Riluzole™, a glutamate release inhibitor, has been approved in the U.S. for the treatment of ALS, and appears to extend the life of at least some patients with ALS. However, some reports have indicated that even though Riluzole™ therapy can prolong survival time, it does not appear to provide an improvement of muscular strength in the patients. Therefore, the effect of Riluzole™ is limited in that the therapy does not modify the quality of life for the patient (Borras-Blasco et al. (1998) Rev. Neurol., 27: 1021-1027).

II. SOD and SOD Mutations

The invention pertains to decreasing the SOD-1 protein (e.g., mutant SOD-1 protein) in cells by reducing or eliminating the expression of the protein with pharmacological modulating agents and their functional analogs. The SOD-1 gene is localized to chromosome 21q22.1. SOD-1 sequences are disclosed in PCT publication WO 94/19493 are oligonucleotide sequences encoding SOD-1 and generally claimed is the use of an antisense DNA homolog of a gene encoding SOD-1 in either mutant and wild-type forms in the preparation of a medicament for treating a patient with a disease. The nucleic acid sequence of human SOD-1 gene can be found at Genbank accession no. NM_{—}000454. The nucleotide sequence of human SOD-1 is also presented in SEQ ID NO: 1. The corresponding SOD-1 protein sequence is presented in SEQ ID NO: 2.

III. Pyrimethamine and its Functional Analogs

In one aspect, the invention pertains to using pyrimethamine and its functional analogs as pharmacological agents that interfere with protein synthesis. Pyrimethamine is an antimalarial drug, that readily penetrates cells in the body and brain. Pyrimethamine has been used for the treatment of malaria, toxoplasmosis, and several other microbial infections (for review see Schweitzer, et al. (1990) FASEB J 4:2441-2452). The antimicrobial effect of pyrimethamine is a result of its inhibition of dihydrofolate reductase (DHFR), and enzymes involved in the folate synthesis pathway. The malaria parasite synthesizes folates de novo whereas the human host must obtain preformed folates and cannot synthesize folate. The inability of the parasite to utilize exogenous folates makes folate biosynthesis a good drug target. DHFR is an ubiquitious enzyme that participates in the recycling of folates by reducing dihydrofolate to tetrahydofolate. The tetrahydrofolate is then oxidized back to dihydrofolate as it participates in biosynthetic reactions (e.g.., thymidylate synthase). Inhibiting DHFR will prevent the formation of thymidylate and lead to an arrest in DNA synthesis and subsequent parasite death. Pyrimethamine is the most common DHFR inhibitor used as an antimalarial.

In one aspect, the invention pertains to lowering SOD-1 expression by administration of pharmacological modulating agents, such a pyrimethamine. Pyrimethamine is a potent inhibitor of SOD-1 expression in the HeLa cell and in the mouse Neuro2A cell lines as shown in the Examples. The mechanism of action for reduction of SOD-1 is not known at this time. Pyrimethamine, however, does not act via dihydrofolate reductase inhibition, because its effects could not be prevented or reversed using folinic acid (the enzymatic product of DHFR). Furthermore, methotrexate, a potent DHFR inhibitor with an unrelated chemical structure, did not reduce SOD-1 protein in the HeLa cell.

While it is not required to provide a mechanism, it is believed that for inhibition of SOD-1 expression, pyrimethamine and its functional analogs putatively act to reduce the intracellular levels of human SOD1 via selective inhibition of protein synthesis, increases in protein disposition, or a decrease in protein stability. Reductions in the amount of mutant SOD1 protein produced would then prevent its neurotoxic effects and ameliorate ALS disease progression.

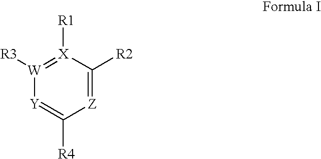

The compounds of the invention are shown in Formula I, above, in which W, X, Y, and Z are independently selected from C, N, O, S, with at least one being non-carbon. R1 through R4 are independently selected from: H, halogen, cyano, SCN, alkyl, haloalkyl, hydroxyalkyl, alkoxyalkyl, aryloxyalkyl, carboxyalkyl, carboalkoxyalkyl, arylalkyl, heteroarylalkyl, cycloalkyl, mercaptoalkyl, alkylthioalkyl, alkylsulfonylalkyl, alkylsulfoxylalkyl, acyl (eg. acetyl), alkenyl, alkynyl, arylalkenyl, arylalkynyl, aryloxyalkenyl, aryloxyalkynyl, arylthioalkenyl, arylthioalkynyl, heteroarylalkenyl, heteroarylalkynyl, heteroaryloxyalkenyl, heteroaryloxyalkynyl, heteroarylthioalkenyl, heteroarylthioalkynyl, aryl, heteroaryl, aroyl (eg. benzoyl), heteroaroyl, and saturated heterocyclyl (eg. morpholino, thiamorpholino, piperazinyl, piperidinyl, pyrrolidinyl, tetrahydrofuranyl), nitro, amino, alkylamino, dialkylamino, arylamino, heteroarylamino, arylalkylamino, heteroarylalkylamino, acylamino, aroylamino, heteroaroylamino, arylguanidino, CONRaryl, CONRheteroaryl, CO₂H, CO₂R, hydroxy or double-bonded oxygen (to satisfy valence), alkoxy, aryloxy, heteroaryloxy, haloalkoxy, carboxyalkoxy, carboalkoxyalkoxy, aryloxy, heteraryloxy, alkenyloxy, alkynyloxy, OCH₂CO₂R, OCH₂CONR₂, mercapto, alkylthio, alkylsulfonyl, alkylsulfoxyl, arylthio, heteroarylthio, arylalkylthio, heteroarylalkylthio, alkenylthio, alkynylthio, SCH₂CO₂R, and SCH₂CONR₂, where R═H, alkyl, haloalkyl, or alkoxyalkyl.

R1, R2, R3, and R4 also may include ring fusions with adjacent positions, giving for example benzo (CH═CH—CH═CH) or saturated groups [eg, (CH₂)n, where n=3-5].

Aryl and heteroaryl groups, either as substituents, parts of other substituents (eg. arylalkyl, heteroarylalkyl), or fused groups, may be optionally substituted with one or more of hydroxy, alkyl, halogen, haloalkyl, alkoxy, haloalkoxy, amino, alkylamino, dialkylamino, acylamino, aroylamino, mercapto, alkylthio, alkylsulfonyl, alkylsulfoxyl, cyano, CO2R, nitro, acyl (eg. acetyl), aroyl (eg. benzoyl).

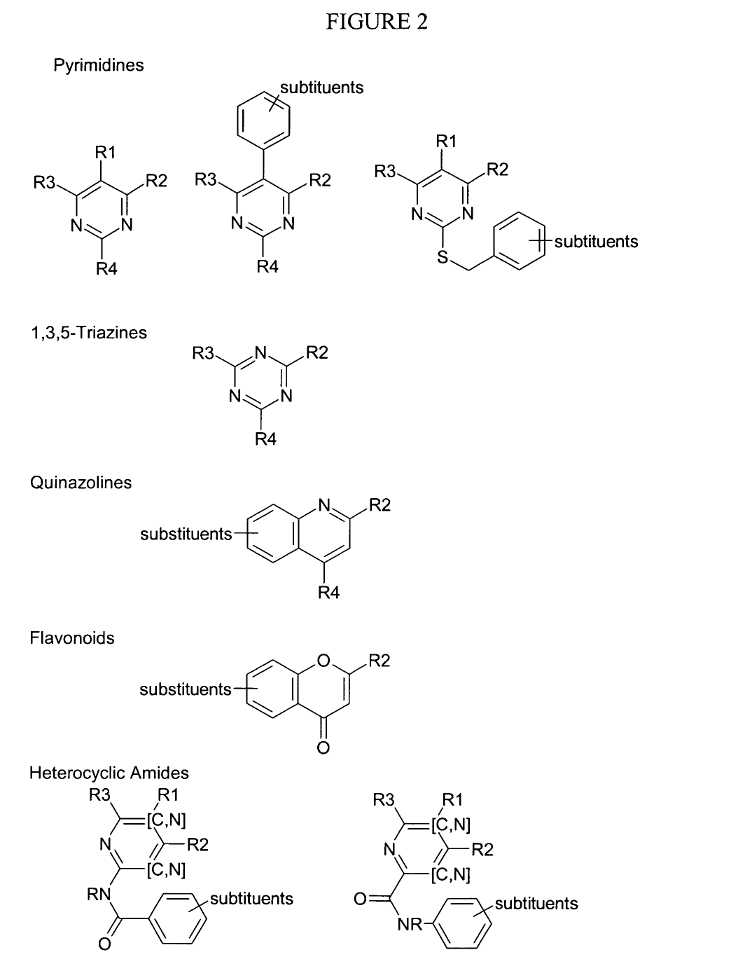

Preferred classes and substituents include pyrimidines, 1,3,5-triazines, flavonoids, quinazolines, 1,2,4-triazines, and heterocyclic amides. Core structures are depicted in FIG. 2, and are described below.

1. Pyrimidines (W,X═C; Y,Z=N), in which R1 is aryl, and R2, R3, and R4 are independently selected from H, hydroxy, mercapto, alkyl, haloalkyl, amino, alkylamino, and dialkylamino. Particularly preferred are compounds in which R1 is halophenyl, R2 and R4 are independently selected from H, amino, and mercapto, and R3 is selected from H, alkyl, and haloalkyl.

A second preferred pyrimidine (W,X═C; Y,Z=N) class includes compounds in which R1 is selected from H, alkyl, and halogen, R2 is selected from OH, alkyl, halogen, and arylamino, R3 is selected from H, alkyl, halogen, dialkylamino, and aryl, and R4 is selected from dialkylamino, halogen, and arylalkylthio.

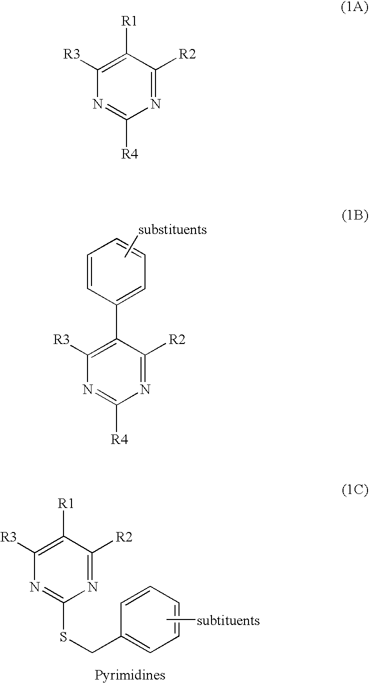

Tables 1A and 1B describes exemplary pyrimidine and pyridine compounds of the present invention and the biological activity thereof.

2. 1,3,5-Triazines (W═C; X,Y,Z=N), in which R2 is selected from OH, NH₂, and haloalkyl, R3 is selected from halogen, haloalkyl, and alkylthio, and R4 is selected from aryl and arylamino.

Particularly preferred are compounds in which R2 is selected from NH₂ and haloalkyl, R3 is alkylthio, and R4 is selected from aryl and arylamino.

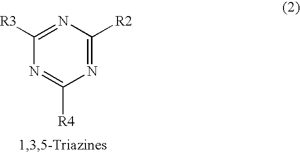

Table 2 describes exemplary triazine compounds of the present invention and the biological activity thereof.

3. Flavonoids (W,Y,Z=C; X═O), in which R2 is selected from H and aryl, R3 is a benzo fused ring, and R4 is a double-bonded oxygen.

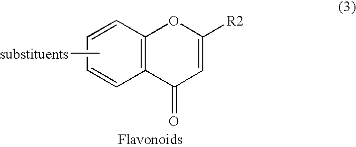

Table 3 describes exemplary flavonoid compounds of the present invention and the biological activity thereof.

4. Quinazolines (W,Y,Z=C; X═N), in which R2 is selected from H, haloalkyl, and alkyl, R3 is a benzo fused ring, and R4 is a saturated heterocycle, eg. piperazine.

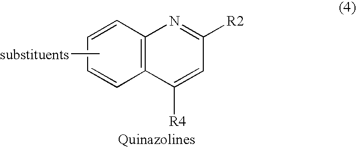

Table 4 describes exemplary quinazolines compounds of the present invention and the biological activity thereof.

5. 1,2,4-Triazines (W,Y,Z=N; X═C), in which R1 is aryl, and R2 and R4 are selected from H, amino, alkyl, haloalkyl, mercapto, alkylthio, and alkoxy.

6. Heterocyclic amides (W═C; X,Z=C,N; Y═N), in which R1, R2, and R3 are selected from H, alkyl, and halogen, and R4 is selected from NRCOaryl and CONRaryl.

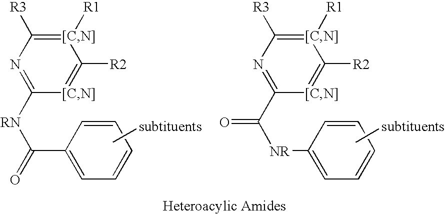

Table 5 describes exemplary heterocyclic amides compounds of the present invention and the biological activity thereof.

IV. Modulation of Neurodegenerative Disorders Using Pharmacological Agents

The role of the nuclear receptor in the neurodegenerative diseases such as ALS, and modulation of the pathway associated with the nuclear receptor maybe a target of a clinical investigation in ALS or other neurodegenerative disease. The data shown in the Examples section indicate that the pyrimethamine and its analogs play a role in decreasing the expression of SOD-1.

The SOD1 G93A (high copy) mouse model for ALS is a suitable mouse that carries 23 copies of the human G93A SOD mutation and is driven by the endogenous promoter. Survival in the mouse is copy dependent. The high copy G93A has a median survival of around 128 days. High molecular weight complexes of mutant SOD protein are seen in the spinal cord beginning around day 30. At day 60 reactive astrocytosis (GFAP reactive) are observed; activated microglia are observed from day 90 onwards. Studies by Gurney et al. showed that at day 90 reactive astrocytosis loses statistical significance while microglial activation is significantly elevated and continues to be elevated through the end stage of the disease (See Gurney, et al. (1996) Ann. Neurol., 39: 147-5739).

Many drugs that have shown efficacy in this model have moved forward into human clinical trials. Experience with riluzole, the only approved drug in the treatment of ALS, indicates that the mouse ALS model is a good predictor of clinical efficacy. Other drugs such as Creatine, Celebrex, Co-enzyme Q10, and Minocycline are under clinical evaluation based on studies in this model.

Mutations in Cu, Zn superoxide dismutase are one of the known causes of familial ALS, and probably for sporatic ALS. Trangenic mice and rats expressing mutant forms of human SOD1 develop motor neuron pathology and clinical symptoms similar to those seen in patients with ALS. The speed of disease progression is dependent on both the number of mutant SOD1 transgenes inserted into the mouse or rat genome and the quantity of protein produced by the transgenes. The more mutant protein created, the more severe the disease phenotype. (Gurney M E, J Neurol Sci. October 1997;152 Suppl 1:S67-73; Alexander G M, Brain Res Mol Brain Res. Nov. 4, 2004;130(1-2):7-15.) The G93A SOD1 transgenic mouse model of ALS is a valid model for the familial form of ALS (Gurney, M E., Science. Jun. 17, 1994;264:1772-5). Targeted reduction of mutant SOD1 using siRNA approaches have demonstrated that the disease can be ameliorated by lowering the quantity of mutant protein in the G93A SOD1 transgenic mouse model of ALS. (Yokota T., Rinsho Shinkeigaku. November 2005;45(11):973-5.; Xia, Neurobiol Dis. September 2006;23(3):578-86. Epub Jul. 20, 2006; Ralph, Nat Med. April 2005;11(4):429-33. Epub Mar. 13, 2005; Raoul, Nat Med. April 2005;11(4):423-8. Epub Mar. 13, 2005) However, the G93A SOD1 mutant is one of more than 100 point mutations in the SOD1 gene that have been identified in patients with ALS. For that reason, targeting individual point mutations in SOD1 would not be a viable therapeutic strategy in a disease such as ALS. Targeted deletion of wild type SOD-1 was not toxic to motoneurons (Guy et al., 2005), therefore overall reduction of SOD-1 should be a useful general strategy to treat SOD1 familial ALS. Therefore, small molecule inhibitors of SOD1 is a preferred approach to treat SOD-1 associated familial ALS, and possibly sporadic disease.

V. Delivery of the SOD1 Inhibiting Pharmacological Agents

The pharmacological agent of the present invention can be incorporated into pharmaceutical compositions suitable for administration to a subject. Typically, the pharmaceutical composition comprises a nuclear receptor modulating pharmacological agent, e.g., pyrimethamine and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. The preferred mode of administration is parenteral (e.g., intravenous, subcutaneous, intraperitoneal, intramuscular). In a preferred embodiment, the pharmacological agent is administered by an intraperitoneal injection.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, (see, for example, Langer, Science 249, 1527 (1990) and Hanes, Advanced Drug Delivery Reviews 28, 97-119 (1997). The agents of this invention can also be administered in the form of a depot injection or implant preparation which can be formulated in such a manner as to permit a sustained or pulsatile release of the active ingredient. The depot injection or implant preparation can, for example, comprise one or more of the pyrimethamine compounds or functional analogs, or comprise a combination of different agents (e.g., pyrimethamine and norethindrone).

The pharmaceutical compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., the pharmacological agent) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The SOD-1 modulating pharmacological agent can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. (See, e.g., Sustained and Controlled Release Drug Delivery Systems, J. R. Robinson, ed., Marcel Dekker, Inc., New York, 1978; U.S. Pat. No. 6,333,051 to Kabanov et al., and U.S. Pat. No. 6,387,406 to Kabanov et al.).

In certain embodiments, a SOD-1 modulating pharmacological agent may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In certain embodiments, a SOD-1 modulating pharmacological agent can be administered in a liquid form. The pharmacological agent should be soluble in a variety of solvents, such as for example, methanol, ethanol, and isopropanol. A variety of methods are known in the art to improve the solubility of the pharmacological agent in water and other aqueous solutions. For example, U.S. Pat. No. 6,008,192 to Al-Razzak et al. teaches a hydrophilic binary system comprising a hydrophilic phase and a surfactant, or mixture of surfactants, for improving the administration of compounds.

Supplementary active compounds can also be incorporated into the compositions. In certain embodiments, a nuclear receptor modulating pharmacological agent can be coformulated with and/or coadministered with one or more additional therapeutic agents that are useful for improving the pharmacokinetics of the pharmacological agent. A variety of methods are known in the art to improve the pharmacokinetics of the pharmacological agent of the present invention. (See e.g., U.S. Pat. No. 6,037,157 to Norbeck et al.).

Other methods of improving the pharmacokinetics of the pharmacological agent have been disclosed, for example, in U.S. Pat. No. 6,342,250 to Masters, U.S. Pat. No. 6,333,051 to Kabanov et al., U.S. Pat. No. 6,395,300 to Straub et al., U.S. Pat. No. 6,387,406 to Kabanov et al., and U.S. Pat. No. 6,299,900 to Reed et al. Masters discloses a drug delivery device and method for the controlled release of pharmacologically active agents. The drug delivery device disclosed by Masters is a film comprising one or more biodegradable polymeric materials, one or more biocompatible solvents, and one or more pharmacologically active agents dispersed uniformed throughout the film. In U.S. Pat. No. 6,333,051, Kabanov et al. disclose a copolymer networking having at least one cross-linked polyamine polymer fragment, at least one nonionic water-soluble polymer fragment, and at least one suitable biological agent, including a pharmacological agent. According to the teachings of this patent, this network, referred to as a nanogel network, improves the therapeutic effect of the pharmacological agent by decreasing side effects and increasing therapeutic action. In another patent, U.S. Pat. No. 6,387,406, Kabanov et al. also disclose another composition for improving the oral delivery of numerous pharmacological agents.

Other methods for improving the delivery and administration of the pharmacological agent include means for improving the ability of the pharmacological agent to cross membranes, and in particular, to cross the blood-brain barrier. In one embodiment, the pharmacological agent can be modified to improve its ability to cross the blood-brain barrier, and in an alternative embodiment, the pharmacological agent can be co-administered with an additional agent, such as for example, an anti-fungal compound, that improves the ability of the pharmacological agent to cross the blood-brain barrier. Alternatively, precise delivery of the pharmacological agent into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for pharmacological agent microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The pharmacological agent can be delivered to regions, such as the cells of the spinal cord, brainstem, or brain that are associated with the disease or disorder. For example, target regions can include the medulla, pons, and midbrain, cerebellum, diencephalon (e.g., thalamus, hypothalamus), telencephalon (e.g., corpus stratium, cerebral cortex, or within the cortex, the occipital, temporal, parietal or frontal lobes), or combinations, thereof.

Pharmacological agents can be used alone or in combination to treat neurodegenerative disorders. For example, the pharmacological agent can be used in conjunction with other existing nuclear receptor modulators, for example, to produce an additive or synergistic effect. Likewise, the pharmacological agent can be used alone or in combination with an additional agent, e.g., an agent which imparts a beneficial attribute to the therapeutic composition, e.g., an agent which effects the viscosity of the composition. The combination can also include more than one additional agent, e.g., two or three additional agents if the combination is such that the formed composition can perform its intended function. In some embodiments, the invention includes administrating a pyrimethamine compound of the present invention, or functional analog thereof, together with for example, at least one progesterone related compound, such as norethindrone, or at least one estrogen related compound, such as estradiol. For descriptions of these compounds and administration, see co-pending applications entitled “Modulation of Neurodegenerative Diseases through the Progesterone Receptor” and “Modulation of Neurodegenerative Diseases through the Estrogen Receptor” filed Mar. 1, 2006, which are incorporated herein in their entirety.

The compounds of the present invention can be conjugated with pharmaceutically acceptable salts to facilitate their long storage and dosing as aqueous solutions. For example, the salt can be derived from a pharmaceutically acceptable acid (e.g., HCl) with or without the use of a pharmaceutically acceptable carrier (e.g., water). Such salts can be derived from either inorganic or organic acids, including for example hydrochloric, hydrobromic, acetic, citric, fumaric, maleic, benzenesulfonic, and ascorbic acids. The pharmaceutical compositions obtained by the combination of the carrier and the salt will generally be used in a dosage necessary to elicit the desired biological effect. This includes its use in a therapeutically effective amount or in a lesser amount when used in combination with other biologically active agents.

The pharmaceutical compositions of the invention may include a “therapeutically effective amount” or a “prophylactically effective amount” of a pharmacological agent of the invention. A “therapeutically effective amount” refers to an amount effective, at dosages and for periods of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a pharmacological agent (e.g., pyrimethamine) is between 5 mg/day to about 200 mg/day administered to a subject, or group of subjects, preferably about 10 mg/day to about 150 mg/day, more preferably about 5 mg/day to about 20 mg/day, and most preferably about 3 mg/day to 10 mg/day. Preferably, administration of a therapeutically effective amount of pharmacological agent (e.g., pyrimethamine), results in a concentration of pharmacological agent in the bloodstream in the range of 1 nanomolar (nM) to 100 millimolar (mM) concentration. For example, a concentration range of about 100 nM to about 10 mM, about, 1 nM to about 1 mM, about 1 nM to about 100 micromolar (μM), about 1 μM to about 500 μM, about 1 μM to about 200 μM or about 10 μM to about 50 μM. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

TABLE 1A
Exemplary pyrimidines and pyridines compounds of the present invention.

| Compound | | | | | | | | |
|---|---|---|---|---|---|---|---|---|
| # | W | X | Y | Z | R1 | R₂ | R₃ | R₄ |
| 1 | C | C | N | N | H | CH₃ | C₆H₅ | NMe₂ |
| 2 | C | C | N | N | H | Cl | NMe₂ | SCH₂C₆H₅ |
| 3 | C | C | N | N | H | CH₃ | CH₃ | SCH₂C₆H₄-3-Br |
| 4 | C | C | N | N | Cl | NHC₆H₄-4-CH₃ | Cl | Cl |
| 5 | C | C | N | N | CH₃ | OH | H | SCH₂C₆H₄-2-Cl |
| 6 | C | C | N | N | C₆H₄-4-Cl | NH₂ | C₂H₅ | NH₂ |
| 7 | C | C | N | N | OC₆H₄-3-CH₃ | OH | H | SH |
| 8 | C | C | N | N | C₆H₄-3-Cl | NH₂ | H | SH |
| 9 | C | C | N | N | H | OH | OH | SCH₂C₆H₄-4-CN |
| 10 | C | C | N | N | C₆H₄-4-OC₃H₇ | NH₂ | H | SH |
| 11 | C | C | N | N | C₆H₄-4-OC₂H₅ | NH₂ | H | SH |
| 12 | C | C | N | N | CH₂C₆H₄-4-Ome | Cl | Cl | Sme |
| 13 | C | C | N | N | Ome | NMe₂ | H | pyrid₂yl-4-OC₃H₇ |
| 14 | C | C | N | N | C₆H₄-2-Cl | NH₂ | H | SH |
| 15 | C | C | N | N | C₆H₄-4-Cl | NH₂ | C₂H₅ | SH |
| 16 | C | C | N | N | C₆H₄-4-Cl | NH₂ | C₂H₅ | CF₃ |
| 17 | C | C | N | N | C₆H₄-4-Cl | CF₃ | C₂H₅ | NH₂ |
| 18 | C | C | N | N | C₆H₄-4-Cl | H | H | SCH₂CCH |
| 19 | C | C | N | N | C₆H₄-3-Cl | H | H | SCH₂CCH |
| 20 | C | C | N | N | C₆H₄-3-Cl | H | H | Sme |
| 21 | C | C | N | N | C₆H₄-4-Cl | H | H | Sme |
| 22 | C | C | N | N | H | CH(CH₃)OC₆H₄- | H | NH₂ |
| | | | | | | 4-Cl | | |
| 23 | C | C | N | N | C₆H₄-4-Cl | NH₂ | H | OH |
| 24 | C | C | N | N | C₆H₄-4-Cl | NH₂ | H | SCH₂CO₂H |
| 25 | C | C | N | N | OC₆H₄-4-CH₃ | OH | H | SH |
| 26 | C | C | N | N | CH═CHCH₂C₆H₄-4-Cl | OH | CH₃ | NH₂ |
| 27 | C | C | N | N | (CH₂)₄C₆H₄-4-OH | NH₂ | CH₃ | NH₂ |
| 28 | C | C | N | N | H | OH | CH₂Spyrimid₂yl- | SH |
| | | | | | | | 3,5-Me₂ | |
| 29 | C | C | N | N | C₆H₄-4-Cl | NH₂ | C₂H₅ | H |
| 30 | C | C | N | N | C₆H₄-4-Cl | NH₂ | C₂H₅ | CH₃ |
| 31 | C | C | N | N | C₆H₄-4-Cl | H | C₂H₅ | NH₂ |
| 32 | C | C | N | N | C₆H₄-4-Cl | CH₃ | C₂H₅ | NH₂ |
| 33 | C | C | N | N | C₆H₄-4-Cl | SH | C₂H₅ | NH₂ |
| 34 | C | C | N | N | C₆H₄-3-Cl | NH₂ | C₂H₅ | SH |
| 35 | C | C | N | N | C₆H₄-2-Cl | NH₂ | C₂H₅ | SH |
| 36 | C | C | N | N | C₆H₄-4-OC₃H₇ | NH₂ | C₂H₅ | SH |
| 37 | C | C | N | N | C₆H₄-4-Cl | C₂H₅ | C₂H₅ | NH₂ |
| 38 | C | C | N | N | C₆H₄-4-Cl | C₂H₅ | C₂H₅ | H |
| 39 | C | C | N | N | C₆H₄-4-Cl | C₂H₅ | C₂H₅ | CH₃ |
| 40 | C | C | N | N | C₆H₄-4-Cl | C₂H₅ | C₂H₅ | CF₃ |
| 41 | C | C | N | N | C₆H₄-4-Cl | C₂H₅ | C₂H₅ | SH |
| 42 | C | C | N | N | C₆H₄-4-Cl | C₂H₅ | C₂H₅ | OH |
| 43 | C | C | N | N | H | NH₂ | C₂H₅ | NH₂ |
| 44 | C | C | N | N | CH₃ | OH | C₂H₅ | NH₂ |
| 45 | C | C | N | N | C₆H₅ | NH₂ | C₂H₅ | NH₂ |
| 46 | C | C | N | N | C₆H₅ | NH₂ | H | NH₂ |
| 47 | C | C | N | N | C₆H₄-4-OC₂H₅ | NH₂ | H | NH₂ |
| 48 | C | C | N | N | C₆H₅ | NMe₂ | H | NH₂ |
| 49 | C | C | N | N | C₆H₄-4-OC₆H₅ | NMe₂ | H | NH₂ |
| 50 | C | C | N | N | C₆H₄-4-Cl | NH₂ | H | H |
| 51 | C | C | N | N | C₆H₄-4-Cl | OH | H | H |
| 52 | C | C | N | N | C₆H₄-4-Cl | NH₂ | C₂H₅ | OH |
| 53 | C | C | N | N | C₆H₄-4-Cl | NH₂ | H | OH |
| 54 | C | C | N | N | C₆H₃₋₃—NO₂-4-Cl | OH | H | NH₂ |
| 55 | C | C | N | C | C₆H₅ | NH₂ | H | NH₂ |
| 56 | C | C | N | C | C₆H₅ | H | H | NH₂ |
| 57 | C | C | N | C | C₆H₄-4-Cl | NH₂ | C₂H₅ | NH₂ |
| 58 | C | C | C | N | C₆H₄-4-Cl | NH₂ | C₂H₅ | NH₂ |
| 59 | C | C | N | C | C₆H₄-4-Cl | H | C₂H₅ | NH₂ |
| 60 | C | C | C | N | C₆H₄-4-Cl | H | C₂H₅ | NH₂ |
| 61 | C | C | N | N | C₆H₄-4-Cl | H | H | Ome |
| 62 | C | C | N | N | C₆H₄-4-Cl | H | H | morpholin4yl |
| 63 | C | C | N | N | C₆H₄-4-Cl | H | H | piperazin1yl |
| 64 | C | C | N | N | C₆H₄-4-Cl | H | H | NHCH₂C₆H₅ |
| 65 | C | C | N | N | C₆H₄-4-Cl | H | H | NHCH₂pyrazin2yl5- |
| | | | | | | | | CH₃ |
| 66 | C | C | N | N | H | NHC₆H₅ | CH₃ | NH₂ |
| 67 | C | C | N | N | H | NHC₆H₄-4-F | NH₂ | NH₂ |
| 68 | C | C | N | N | H | NHC₆H₄-4-Ome | NH₂ | NH₂ |
| 69 | C | C | N | N | H | NHC₆H₄-4-Cl | NH₂ | NH₂ |
| 70 | C | C | N | N | CN | NHC₆H₄-4-Cl | NH₂ | NH₂ |
| 71 | C | C | N | N | H | NHC₆H₄-4-Cl | CH₃ | NH₂ |
| 72 | C | C | N | N | C₂H₅ | NH₂ | C₂H₅ | NH₂ |
| 73 | C | C | N | N | C₆H₄-4-CH₃ | NH₂ | C₂H₅ | NH₂ |
| 74 | C | C | N | N | C₆H₄-3-CH₃ | NH₂ | C₂H₅ | NH₂ |
| 75 | C | C | N | N | C₆H₄-2-CH₃ | NH₂ | C₂H₅ | NH₂ |
| 76 | C | C | N | N | C₆H₃-2,3-Me₂ | NH₂ | C₂H₅ | NH₂ |
| 77 | C | C | N | N | pyrid₄yl | NH₂ | C₂H₅ | NH₂ |
| 78 | C | C | N | N | pyrid₃yl | NH₂ | C₂H₅ | NH₂ |
| 79 | C | C | N | N | pyrid₂yl | NH₂ | C₂H₅ | NH₂ |
| 80 | C | C | N | N | pyrrol₅yl | NH₂ | C₂H₅ | NH₂ |
| 81 | C | C | N | N | C₆H₄-4₄-Cl | NH₂ | CH(CH₃)₂ | NH₂ |
| 82 | C | C | N | N | C₆H₄-4-Cl | NH₂ | C₃H₇ | NH₂ |
| 83 | C | C | N | N | C₆H₄-4-Cl | NH₂ | CH₃ | NH₂ |
| 84 | C | C | N | N | C₆H₄-4-Cl | OH | C₂H₅ | NH₂ |
| 85 | C | C | N | N | C₆H₄-4-Cl | H | C₂H₅ | H |
| 86 | C | C | N | N | CH₃ | NH₂ | C₂H₅ | NH₂ |
| 87 | C | C | N | N | C₃H₇ | NH₂ | C₂H₅ | NH₂ |
| 88 | C | C | N | N | OC₆H₄-4-Cl | OH | H | Sme |
| 89 | C | C | N | N | H | OH | C₆H₃-3,4-(Ome)₂ | H |
| 90 | C | C | N | N | C₆H₄-4-Cl | NH₂ | CF₃ | NH₂ |
| 91 | C | C | N | N | C₆H₄-4-Cl | NH₂ | CF₃ | SH |
| 92 | C | C | N | N | H | SC₆H₄-4-NH₂ | Cl | NH₂ |
| 93 | C | C | N | N | H | CH₃ | CH₃ | NHC(═NH)NHC₆H₄- |
| | | | | | | | | 3-Cl |
| 94 | C | C | N | N | H | CH₃ | CH₃ | SC₆H₄-4-NH₂ |
| 95 | C | C | N | N | CN | Sme | C₆H₄-4-Cl | NH₂ |
| 96 | C | C | N | N | C₂H₅ | OH | C₆H₄-4-CH₃ | H |
| 97 | C | C | N | N | (CH₂)₃C₆H₄-4-Sme | NH₂ | CH₃ | NH₂ |
| 98 | C | C | N | N | CH₂C₆H₄-4-Ome | OH | CH₃ | NH₂ |
| 99 | C | C | N | N | H | OH | H | SCH₂C₆H₃-2,4-Cl₂ |
| 100 | C | C | N | N | (CH₂)₃C₆H₄-4-Ome | NH₂ | CH₃ | NH₂ |
| 101 | C | C | N | N | H | OH | H | SCH₂C₆H₃-2-Cl-₆—F |
| 102 | C | C | N | N | CH₂C₆H₃-3,4-(Ome)₂ | NH₂ | H | NH₂ |
| 103 | C | C | N | N | Cl | NEt₂ | C₆H₅ | H |
| 104 | C | C | N | N | CCCH₂C₆H₅ | NH₂ | CH₃ | NH₂ |
| 105 | C | C | N | N | H | OH | H | SCH₂C₆H₄-4-CN |
| 106 | C | C | N | N | CN | Cl | NHC₆H₅ | Cl |
| 107 | C | C | N | N | C(O)CH₃ | NH₂ | CH₃ | C₆H₄-4-Cl |
| 108 | C | C | N | N | CCC₆H₅ | NH₂ | CH₃ | NH₂ |
| 109 | C | C | N | N | CH═CHC₆H₅ | NH₂ | CH₃ | NH₂ |
| 110 | C | C | N | N | OCH₂C₆H₃-2,4-Cl₂ | NH₂ | CH₃ | NH₂ |
| 111 | C | C | N | N | OCH₂CH₂C₆H₄-4-Cl | NH₂ | CH₃ | NH₂ |
| 112 | C | C | N | N | C₆H₃-3,4-Cl₂ | NH₂ | CH₃ | NH₂ |
| 113 | C | C | N | N | H | NH₂ | OCH₃ | SCH₂C₆H₄-2-Cl |
| 114 | C | C | N | N | H | OH | CH₃ | SCH₂C₆H₃-2,4-Cl₂ |
| 115 | C | C | N | N | CH₃ | Net₂ | C₆H₅ | H |
| 116 | C | C | N | N | CH₂C₆H₄-4-Ome | OH | OH | SH |
| 117 | C | C | N | N | H | CH₃ | CH₃ | OCH₂C₆H₄-3-Cl |
| 118 | C | C | N | N | (CH₂)₃C₆H₄-2-CH₃ | NH₂ | CH₃ | NH₂ |
| 119 | C | C | N | N | C₆H₅ | H | SCH₂CO₂H | NH₂ |
| 120 | C | C | N | C | H | CH₃ | H | NHCH₂C₆H₃-2-OH- |
| | | | | | | | | 5-Br |

TABLE 1B
Biological Activity (% inhibition at 10 uM) of Pyrimidines
and Pyridines listed in Table 1A.

| Compound # | Biological Activity |
|---|---|
| from Table 1A | (% inhibition at 10 μM) |
| 1 | 33 |
| 2 | 77 |
| 3 | 88 |
| 4 | 96 |
| 5 | 79 |
| 6 | 50 |
| 7 | 94 |
| 8 | 99 |
| 9 | 58 |
| 10 | 99 |
| 11 | 99 |
| 12 | 40 |
| 22 | 84 |
| 23 | 99 |
| 24 | 99 |
| 25 | 97 |
| 26 | 97 |
| 27 | 95 |
| 28 | 98 |
| 88 | 90 |
| 89 | 84 |
| 92 | 81 |
| 93 | 78 |
| 94 | 77 |
| 95 | 76 |
| 96 | 70 |
| 97 | 69 |
| 98 | 67 |
| 99 | 66 |
| 100 | 65 |
| 101 | 65 |
| 102 | 64 |
| 103 | 61 |
| 104 | 60 |
| 105 | 53 |
| 106 | 53 |
| 107 | 52 |
| 108 | 49 |
| 109 | 38 |
| 110 | 38 |
| 111 | 37 |
| 112 | 36 |
| 113 | 36 |
| 114 | 35 |
| 115 | 34 |
| 116 | 33 |
| 117 | 33 |
| 118 | 31 |
| 119 | 30 |
| 120 | 79 |

TABLE 2
Exemplary 1,3,5 triazines compounds of the present invention.

| | Biological | | | | | | | | |
|---|---|---|---|---|---|---|---|---|---|
| | Activity | | | | | | | | |
| | (% | | | | | | | | |
| | inhibition | | | | | | | | |
| Compound | at 10 μM) | W | X | Y | Z | R1 | R2 | R3 | R4 |
| 164 | 43 | C | N | N | N | — | NH₂ | Cl | NHC₆H₄-4-CH₃ |
| 165 | 99 | C | N | N | N | — | NH₂ | CH₂Cl | NHC₆H₃-2,4-Me₂ |
| 166 | 98 | C | N | N | N | — | CHCl₂ | SC₂H₅ | C₆H₅ |
| 167 | | C | N | N | N | — | OH | SC₂H₅ | C₆H₄-4-Cl |
| 168 | 75 | C | N | N | N | — | CHCl₂ | SCH₃ | C₆H₄-4-CH₃ |
| 169 | | C | N | N | N | — | CHCl₂ | SCH₃ | C₆H₄-4-Br |
| 170 | 99 | C | N | N | N | — | NH₂ | NHC₄H₉ | C₆H₃-2-OH-5-Cl |
| 171 | | C | N | N | N | C₆H₄-4-Cl | NH₂ | C₂H₅ | NH₂ |
| 172 | | C | N | N | N | — | NH₂ | NH₂ | C₆H₄-4-Cl |
| 173 | | C | N | N | N | — | NH₂ | NH₂ | C₆H₃-2-Ome-5-Cl |
| 174 | | C | N | N | N | — | NH₂ | NH₂ | C₆H₃-2-OH-5-Cl |
| 175 | | C | N | N | N | — | NH₂ | NHCH₃ | C₆H₃-2-OH-5-Cl |
| 176 | | C | N | N | N | — | NH₂ | NH₂ | CH₂C₆H₄-4-Cl |
| 177 | | C | N | N | N | — | NH₂ | NH₂ | CH₂C₆H₅ |
| 178 | | C | N | N | N | — | NH₂ | H | NHC₆H₃-3-Cl-4-F |
| 179 | | C | N | N | N | — | NH₂ | H | NHC₆H₄-4-Cl |
| 180 | | C | N | N | N | — | NH₂ | NH₂ | SCH₂C₆H₄-4-Cl |
| 181 | | C | N | N | N | — | NH₂ | NH₂ | OCH₂C₆H₄-3-Cl |
| 182 | | C | N | N | N | — | NH₂ | NH₂ | SC₆H₄-4-F |
| 183 | | C | N | N | N | — | NH₂ | NH₂ | OC₆H₄-4-Cl |
| 184 | | C | N | N | N | — | NH₂ | NH₂ | NHCH₂C₆H₄-4-Cl |
| 185 | | C | N | N | N | — | NH₂ | CHCl₂ | NHC₆H₃-2,4-Cl₂ |
| 186 | 82 | C | N | N | N | — | Cl | C₆H₅ | NHCH(CH₃)₂ |
| 187 | 77 | C | N | N | N | — | NH₂ | CH₂Cl | NHC₆H₅ |
| 188 | 38 | C | N | N | N | — | NH₂ | Cl | NHCH(CH₃)C₆H₅ |
| 189 | 35 | C | N | N | N | — | NH₂ | NHCH₃ | C₆H₃-2-OMe-5-Cl |
| 190 | 34 | C | N | N | N | — | NH₂ | NH₂ | C₆H₄-2-Cl |
| 191 | 32 | C | N | N | N | — | NH₂ | NHC₃H₇ | C₆H₃-2-OMe-5-Cl |
| 192 | 32 | C | N | N | N | — | NH₂ | NH₂ | C₆H₄-3-NO₂ |
| 193 | 29 | C | N | N | N | — | NH₂ | CHCl₂ | NHC₆H₄-4-Cl |

TABLE 3
Exemplary flavonoids compounds of the present invention.

| | Biological | | | | | | | | |
|---|---|---|---|---|---|---|---|---|---|
| | Activity | | | | | | | | |
| Compound | (% inhibition | | | | | | | | |
| # | at 10 μM) | W | X | Y | Z | R1 | R2 | R3 | R4 |
| 121 | 71 | C | O | C | CH | — | C₆H₄-2-OH | CH═CH— | ═O |
| | | | | | | | | CH═C(Ome) | |
| 122 | | C | O | C | CH | — | C₆H₄-4- | CH═CH— | ═O |
| | | | | | | | Ome | CH═C(Ome) | |
| 123 | | C | O | C | CH | — | C₆H₄-3- | CH═CH— | ═O |
| | | | | | | | Ome | C(Ome)═CH | |
| 124 | | C | O | C | CC₆H₄-4- | — | H | C(Ome)═C(OH)— | ═O |
| | | | | | Ome | | | CH═CH | |
| 125 | | C | O | C | CC₆H₄-4- | — | H | CH═C(Ome)— | ═O |
| | | | | | Ome | | | CH═CH | |
| 126 | | C | O | C | CC₆H₅ | — | H | CH═C(OH)— | ═O |
| | | | | | | | | CH═C(OH) | |
| 127 | | C | O | C | CH | — | C₆H₅ | C(OH)═C(OH)— | ═O |
| | | | | | | | | CH═CH | |
| 128 | | C | O | C | CH | — | C₆H₃-3,4- | CH═CH—CH═CH | ═O |
| | | | | | | | OMe₂ | | |
| 129 | | C | O | C | COMe | — | C₆H₅ | CH═C(Ome)— | ═O |
| | | | | | | | | CH═CH | |
| 130 | | C | O | C | CH | — | C₆H₅ | CH═CH—CH═CH | ═O |
| 131 | | C | O | C | CH | — | C₆H₄-4-Cl | CH═CH—CH═CH | ═O |
| 132 | | C | O | C | CC₆H₄-4- | — | H | CH═CH—CH═CH | ═O |
| | | | | | Ome | | | | |
| 133 | | C | O | C | CC₆H₄-4-Cl | — | H | CH═CH—CH═CH | ═O |
| 134 | | C | S | C | CC₆H₄- | — | H | CH═CH—CH═CH | ═O |
| | | | | | Ome | | | | |
| 135 | | C | S | C | CH | — | C₆H₅ | CH═CH—CH═CH | ═O |

TABLE 4
Exemplary quinazolines compounds of the present invention.

| | Biological | | | | | | | | |
|---|---|---|---|---|---|---|---|---|---|
| | Activity | | | | | | | | |
| | (% | | | | | | | | |
| Compound | inhibition | | | | | | | | |
| # | at 10 μM) | W | X | Y | Z | R1 | R2 | R3(Y) | R4 |
| 136 | 64 | C | N | C | CH | — | CF₃ | CH═CH—CF═CH | piperazin1yl-4-methyl |
| 137 | | C | N | C | CH | — | CF₃ | CH═CH—CH═CH | piperazin1yl-4-methyl |
| 138 | | C | N | C | CH | — | CH₃ | CH═CH—CF═CH | piperazin1yl-4-methyl |
| 139 | | C | N | C | CH | — | CH₃ | CH═CH—CH═CH | piperazin1yl-4-methyl |
| 140 | | C | N | C | CH | — | CF₃ | CH═CH—CH═CH | piperazin1yl-4-benzyl |
| 141 | | C | N | C | CH | — | CF₃ | CH═CH—CH═CH | piperazin1yl |
| 142 | | C | N | C | CH | — | CF₃ | CH═CH—CH═CH | CH₂C₆H₅ |
| 143 | | C | N | C | CH | — | CF₃ | CH═CH—CH═CH | CH₂C₆H₄-4-Cl |
| 144 | | C | N | C | CH | — | CF₃ | CH═CH—CH═CH | CH₂C₆H₄-3-OC₂H₅ |
| 145 | | C | N | C | CH | — | CF₃ | CH═CH—CH═CH | NHCH₂C₆H₅ |
| 146 | | C | N | C | CH | — | CF₃ | CH═CH—CH═CH | NHCH₂C₆H₄-4-F |
| 147 | | C | N | C | CH | — | CF₃ | CH═CH—CH═CH | morpholin4yl |
| 148 | | C | N | C | CH | — | CF₃ | CH═CH—CH═CH | piperidin1yl |
| 149 | | C | N | C | CH | — | CF₃ | CH═CH—CH═CH | OCH₂C₆H₅ |
| 150 | | C | N | C | CH | — | CF₃ | CH═CH—CH═CH | OC₆H₄-4-Cl |
| 151 | | C | N | C | CH | — | CF₃ | CH═CH—CH═CH | NHC₃H₇ |
| 152 | | C | N | C | CH | — | CF₃ | CH═CH—CH═CH | C(O)C₆H₅ |
| 153 | | C | N | C | CH | — | H | CH═CH—CH═CH | piperazin1yl-4-methyl |
| 154 | | C | N | C | CH | — | H | CH═CH—C(Cl)═CH | piperazin1yl-4-methyl |
| 155 | | C | N | C | CH | — | CH₃ | CH═CH—C(Cl)═CH | piperazin1yl-4-methyl |
| 156 | | C | N | C | CH | — | CH₃ | CH═CH—C(OMe)═CH | piperazin1yl-4-methyl |
| 157 | | C | N | C | CH | — | CH₃ | CH═CF—CH═CH | piperazin1yl-4-methyl |
| 158 | | C | N | C | CH | — | CH₃ | CF═CH—CH═CH | piperazin1yl-4-methyl |
| 159 | | C | N | C | CH | — | CH₃ | CH═CH—CH═CF | piperazin1yl-4-methyl |
| 160 | | C | N | C | CH | — | CF₃ | CH═CH—CH═CH | piperazin1yl-4-phenyl |
| 161 | | C | N | C | CH | — | CF₃ | CH═CH—CH═CH | SCH₂C₆H₅ |
| 162 | | C | N | C | CH | — | CF₃ | CH═CH—CH═CH | SCH₂C₆H-4-Cl |
| 163 | | C | N | C | CH | — | CF₃ | CH═CH—CH═CH | SC₆H₄-4-Cl |

TABLE 5
Exemplary heterocyclic amides compounds of the present invention.

| | Biological | | | | | | | | |
|---|---|---|---|---|---|---|---|---|---|
| | Activity | | | | | | | | |
| | (% | | | | | | | | |
| Compound | inhibition | | | | | | | | |
| # | at 10 μM) | W | X | Y | Z | R1 | R2 | R3 | R4 |
| 194 | 92 | C | C | N | N | H | H | H | NHC(O)C₆H₄-4-NH₂ |
| 195 | 91 | C | N | N | C | — | CH₃ | Cl | C(O)NHC₆H₄-4-Cl |
| 196 | 88 | C | N | N | C | — | CH₃ | Cl | C(O)NHC₆H₅ |
| 197 | 74 | C | C | N | CCl | H | H | H | NHC(O)C₆H₄-4-CH₃ |
| 198 | 67 | C | C | N | CH | H | CH₃ | H | NHC(O)C₆H₄-3-Cl |
| 199 | 94 | C | C | N | CH | CH₃ | H | H | NHC(O)C₆H₃-2,5-Cl₂ |
| 200 | 39 | C | C | N | CH | Cl | H | H | NHC(O)C₆H₃-3,4-Me₂ |
| 201 | 39 | C | C | N | CH | Cl | H | H | NHC(O)C₆H₄-3-CH₃ |
| 202 | 67 | C | C | N | N | NHC(O)C₆H₅ | OH | CH₃ | OH |
| 203 | | C | C | N | CH | Cl | H | H | NHC(O)C₆H₃-2,5-Cl₂ |
| 204 | | C | C | N | CH | Cl | H | H | NHC(O)C₆H₄-4-Cl |
| 205 | | C | C | N | CH | Cl | H | H | NHC(O)C₆H₃-2,4-Cl₂ |
| 206 | | C | C | N | CH | CH₃ | H | H | NHC(O)C₆H₃-2,4-Cl₂ |
| 207 | | C | C | N | CH | CH₃ | H | H | NHC(O)C₆H₄-4-Cl |
| 208 | | C | C | N | CH | H | H | H | NHC(O)C₆H₃-2,5-Cl₂ |
| 209 | | C | C | N | CH | H | H | H | NHC(O)C₆H₄-4-Cl |
| 210 | | C | C | N | CH | H | H | H | NHC(O)C₆H₃-2,4-Cl₂ |
| 211 | | C | C | N | CH | H | CH₃ | H | NHC(O)C₆H₃-2,4-Cl₂ |
| 212 | | C | C | N | CH | H | CH₃ | H | NHC(O)C₆H₄-4-Cl |
| 213 | | C | C | N | CH | H | H | Cl | NHC(O)C₆H₃-2,5-Cl₂ |
| 214 | | C | C | N | CH | H | H | Cl | NHC(O)C₆H₄-4-Cl |
| 215 | | C | C | N | CH | H | H | Cl | NHC(O)C₆H₃-2,4-Cl₂ |
| 216 | | C | C | N | CH | H | H | CH₃ | NHC(O)C₆H₃-2,4-Cl₂ |
| 217 | | C | C | N | CH | H | H | CH₃ | NHC(O)C₆H₄-4-Cl |
| 218 | | C | C | N | N | CH₃ | H | H | NHC(O)C₆H₃-2,5-Cl₂ |
| 219 | | C | C | N | N | Cl | H | H | NHC(O)C₆H₃-2,5-Cl₂ |
| 220 | | C | C | N | N | H | H | H | NHC(O)C₆H₃-2,5-Cl₂ |
| 221 | | C | C | N | N | H | C₂H₅ | H | NHC(O)C₆H₃-2,5-Cl₂ |
| 222 | | C | C | N | N | H | CH₃ | CH₃ | NHC(O)C₆H₃-2,5-Cl₂ |

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Materials and Methods

(i) Cell Culture:

The human cervical carcinoma derived HeLa cell line (ATCC) was found to express SOD-1 protein and mRNA and was used as the model system to identify compounds that inhibit SOD-1 expression. Briefly, cells were maintained in Dulbecco's Minimal Essential Medium, with high glucose, supplemented with glutamine, 4 mM, certified fetal bovine serum, 10%, and penicillin, streptomycin, and nystatin (all from Invitrogen). Incubation conditions were 37 degrees and 99% relative humidity, with CO₂ at 5%. Cultures were passaged when they reached 90% confluence. For pharmacological experiments, cells were plated into sterile tissue culture treated 96 well plates at a density of 3,500 cells/well in 150 μl medium.

(ii) Pharmacological Agents:

All compounds were dissolved in 100% DMSO, at a stock concentration of 10 mM. Compounds 1, 5, 8, 22, 23, 24, 92, 101, 102, 119, 136, 164, 170, 187, 189, 190, 191, and 192 were obtained from Analytical Services of Delaware Inc. (Newark, Del.). Compounds 18-21 were obtained from Bionet. Compounds 7, 25, 26, 27, 88, 89, 94, 95, 96, 97, 100, 103, 104, 106, 108, 109, 110, 111, 112, 115, 116 117, 118, 186, 188, 193, 194, 195, and 196 were obtained from FMC Corporation (Philadelphia, Pa.). Compounds 6 and 121 through 135 were obtained from Microsource Discovery (Gaylordsville, Conn.). Compounds 2, 3, 4, 9, 10, 11, 12, 28, 93, 98, 99, 105, 107, 113, 114, 120, 165, 166, 197, 198, 199, 200, 201, and 202 were obtained from Princeton Biomolecular Research (Monmouth Junction, N.J.). Compound 167 was obtained from Ryan Scientific. Compound 169 was obtained from Spex Inc (Wakefield, R.I.).

Compounds 13, 14, 15, 16, 17, and 36 were synthesized using the representative novel synthesis methods described below.

### Preparation of 2-mercapto-4-amino-5-(4-chlorophenyl)-6-ethylpyrimidine, Compound 15 (Table 1A)

Under an atmosphere of dry nitrogen, an ice-cooled solution of 1.5 g (10 moles) of 4-chlorophenylacetonitrile and 1.6 g (16 mmoles) of ethyl propionate in 100 mL of anhydrous tetrahydrofuran was treated with 2.5 g (22 mmoles) of solid potassium tert-butoxide over 15 min using a powder addition funnel. The solution was allowed to warm to room temperature and kept at room temperature overnight. The yellow solution was poured into excess 0.1N HCl and extracted with ethyl acetate. The organic layer was separated and washed with 10% aqueous lithium chloride solution followed by brine, then dried over anhydrous sodium sulfate. Evaporation of solvent left Intermediate I, 2-(4-chlorophenyl)-3-oxovaleronitrile, as a yellow oil, 2.0 g, suitable for the next reaction.

Under dry nitrogen, sodium metal, 0.5 g (22 g-atoms) was dissolved in 50 mL of absolute ethanol. The 3-oxonitrile above plus 1.5 g (20 mmoles) of thiourea was added to the resulting sodium ethoxide solution and the mixture refluxed overnight. Careful neutralization with 0.1N HCl caused precipitation of pure Compound 15, 1.5 g (56% yield), as a white solid.

Preparation of 2-mercapto-4-amino-5-(4-propoxyphenyl)-6-ethylpyrimidine, Compound 36 (Table 1A). In the same manner, Compound 36 was prepared from 4-propoxybenzonitrile in 50% yield as a white solid.

Preparation of 2-trifluoromethyl-4-amino-5-(4-chlorophenyl)-6-ethylpyrimidine, Compound 16 (Table 1A) Under dry nitrogen, sodium metal, 0.8 g (35 g-atoms) was dissolved in 75 mL of absolute ethanol. Intermediate I, above, 2.0 g (10 mmol) plus 1.5 g (10 mmoles) of trifluoroacetamidine hydrochloride was added to the sodium ethoxide solution and the mixture was refluxed overnight. The product was precipitated by addition of water, and the crude white solid was recrystallized from aqueous ethanol to provide Compound 16, 1.5 g (50% yield) as a white solid.

(iii) Experimental Protocol:

After plating and 6 hours for attachment, drugs were added to the medium in a concentration of 10 μM. Following 72 hours of incubation with the drugs, the cells were photographed at 100× using an inverted microscope and digital camera, so that cytotoxicity could be evaluated. After photodocumentation, the medium was removed and the cells were washed once with phosphate buffered saline, and then 50-100 μl molecular biology grade water containing a protease inhibitor cocktail was added. After 10 min incubation, the plates were placed in −80 degrees to induce complete lysis. Plates were then thawed and 4-25 μl was transferred from each well into a maxsorp ELISA plate coated with anti-human SOD-1 antibody, which contained 75-100 μl phosphate buffered saline or blocking buffer. A second antibody pair (a polyclonal anti-SOD-1/HRP conjugated goat anti-rabbit) was then added to the well, and incubation was conducted for 1 hour at room temperature. At the conclusion of the incubation, the plate was washed three times (wash buffer from KPL Inc.) and Sure Blue Reserve HRP Substrate was added. Following a 5-10 min incubation, the reaction (which had turned blue to varying degrees) was stopped by the addition of a stop reagent (KPL). The plate was then shaken gently for 5 seconds and the absorbance at 450 nm read on a Tecan Plate reader. Absorbance from each sample were compared to standard curve of purified recombinant human SOD-1 assayed on the same ELISA plate, and SOD-1 immunoreactivity (ng/ml) was estimated by comparison with the standard curve.

(iv) Bradford Protein Assay:

To determine if decrements found in the SOD-1 assay were simply the result of cytotoxic effects of the drug treatment, total protein was determined for each well. While the ELISA incubation was ongoing, 10 μl of the remaining lysate was removed from each well and placed into another empty plate, and BioRad Bradford reagent (100 μl) was added to the protein. After a 15 min incubation at room temperature the plate was shaken gently for 5 seconds and the absorbance was read at 595 nm in a Tecan Sunrise plate reader. Protein concentrations in each well were thus determined by comparison with protein standards that were run on the same plate.

(v) Quantitative RT-PCR:

HeLa cells at 3500 cells/well in a 96 well plate were treated with a compound of the present invention for 72 h as above and then cells were lysed and total RNA extracted using the Gentra RNA extraction protocol and reagents. The purified RNA was then used as the template in a reverse transcription reaction using Superscript III MMLV Transcriptase primed with oligoDT. A PCR reaction was performed on the resultant cDNA to amplify the cDNA corresponding to human SOD-1, human TATA-box binding protein, and human Beta-2 microglobulin. The PCR reactions were run in separate tubes for 20, 25, and 30 cycles and the amplicons were then run on a 2% agarose gel containing ethidium bromide. The fluorescence emitted by the ethidium bromide stained bands following stimulation by a UV light source was captured using a digital camera. The digitized images were analyzed using ImageJ (NIH) and the bands for SOD-1 were compared with the bands for TATA-box binding protein and Beta2 Microglobulin (these housekeeping genes were unaffected by the drugs) while in the linear range of cycles, 25 cycles under these conditions, for increases or decreases relative to controls.

(vi) GeneChip Experiments:

Total cellular mRNA was prepared from HeLa cells with or without treatment using a Qiagen RNA mini kit followed by oligotex mRNA mini kit. Double-stranded cDNAs were synthesized from 2 μg total mRNA using the Superscript Choice System for cDNA synthesis (Invitrogen) with the T7-(dT)24 primer following the manufacturer's recommendations. cDNAs were cleaned up by phase lock gel (PLG) phenol/chloroform extraction and concentrated by ethanol precipitation. Biotin-labeled cRNA was synthesized from cDNA by in vitro transcription using the Bioarray HighYield RNA transcript Labeling Kit (Affymetrix) following vendor's recommendation. In vitro transcription products were cleaned up using RNeasy spin columns (Qiagen) and fragmented by metal-induced hydrolysis in fragmentation buffer (40 mM Tris-acetate, pH 8.1, 100 mM KOAc, 30 mM MgOAc). Fragmented cRNA was then subjected to Affymetrix GeneChip sets in hybridization buffer (100 mM MES, 1M NaCl, 20 mM EDTA, 0.01% Tween-20). GeneChip images were analyzed with Affymetrix Microarray Suite V5.0 and Affymetrix Data Mining Tool V3.0. Signal intensities of all probe sets were scaled to a target value of 150. Results of Detection Call, Change Call and Signal Log Ratio were obtained by applying the default parameters to statistical algorithms for both absolute and comparison analyses.

(vii) Western Blotting.

Animals were overdosed with sodium pentobarbital (250 mg/kg, i.p.). Spinal cords were dissected and homogenized in 20 mM Tris-HCl, pH 7.5, 2 mM DTT, 0.1 mg of leupeptin, 1 mM EDTA, and 1 mM EGTA. The homogenate was then centrifuged at 14,000×g to pellet debris. Protein concentration was measured using the BCA protein assay (Pierce, Rockford, Ill.). Protein (10 μg) from each sample was run on a 4-20% Bis-Tris gel (Invitrogen, San Diego, Calif.). After transfer, membranes were washed in PBS, followed by overnight incubation in blocking buffer (1% protease free BSA (Sigma), PBS, and 0.1% Tween 20). The membrane was then probed with a polyclonal sheep anti-human SOD1 (Sigma) antibody at 1:10,000 dilution. After several washes, membranes were incubated with an horseradish peroxidase (Sigma) or IR dye (Li-Cor, Lincoln, Nebr.)—conjugated goat or donkey anti sheep secondary antibody (1:10,000 in blocking buffer), and the immunoreactive signals were visualized using a Li-Cor Odyssey infrared scanner or incubated with a HRP substrate and scanned using a conventional scanner (see below) and analyzed using the Li-Cor software or NIH Image for quantitation of band density.

(viii) Dot Blotting.

Brain tissue was added to 9 volumes of purified water containing TritonX (1%) and protease inhibitor.—i.e.: 180 mg brain tissue to 1620 ul water). The sample was then homogenized with a sonicator at 50% duty and 70% power for 10 seconds. The sample was then centrifuged @ 10,000 RPM for 10 min, and the supernatant collected. The sample was then boiled for 10 min, and promptly spotted at a volume of 10 μl onto a PVDF or nitrocellulose membrane (Millipore). The membrane was then dried and placed in blocking buffer (1% bovine serum albumin in pH 7.4 phosphate buffered 0.9% saline) overnight at 4 degrees C. The membrane is then washed for 30 min in wash buffer (KPL, Gaithersburgh, Md.) and then placed in blocking buffer containing rabbit anti alpha-synuclein antibody (Sigma) diluted at 1:10,000 for one hour with constant agitation. The membrane is washed again for 30 min before incubation in blocking buffer containing a HRP labeled goat or donkey anti rabbit antibody. After one hour incubation with the anti-rabbit antibody, the membrane is washed again for 30 min, dipped briefly in purified water, and placed in a HRP substrate solution (KPL) until the spots become visible. The membrane is then dried, and an image digitized with a typical flat bed scanner and the spots are quantified by comparison with a standard curve using an image analysis program such as NIH Image J.

### Example 2

### Testing the Biological Activity of the Compounds

This example describes how to examine the in vitro effects of the antimalarial drug, pyrimethamine, as well as the compounds listed in Tables 1 through 5, on SOD-1 activity. The human cervical carcinoma derived HeLa cell line (ATCC) were cultured in Dulbecco's Minimal Essential Medium, with high glucose, supplemented with glutamine, 4 mM, certified fetal bovine serum, 10%, and penicillin, streptomycin, and nystatin (all from Invitrogen). Incubation conditions were 37° C. and 99% relative humidity, with CO₂ at 5%. Cultures were passaged when they reached 90% confluence. For pharmacological experiments, cells were plated into sterile tissue culture treated 96 well plates at a density of 3,500 cells/well in 150 μl medium.

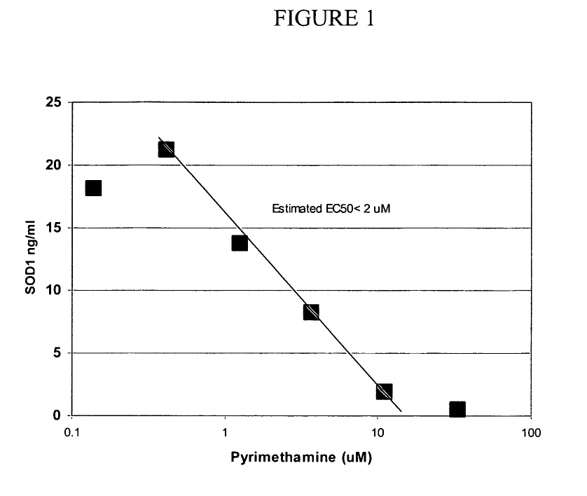

Following 72 hours of incubation with each of the compounds, the cells were photographed and processed as described in Example 1 (iii). The total protein of the lysates was determined by Bradford assay as described in Example 1 (iv). The results of this study with pyrimethamine are shown in FIG. 1. These results show that pyrimethamine added to culture medium of HeLa cells 72 hours before harvest significantly reduced the levels of SOD-1 protein, while total protein levels were unaffected. This reduction was dose related and maximal by 10 μM, with an IC₅₀ of less than 3 μM. Pyrimethamine (5 μM) caused a dose-related decrease in hSOD-1 mRNA in HeLa cells following 72 h treatment. The biological activity results recorded in Tables 1B, 2, 3, 4, and 5 show the % reduction of hSOD-1 protein levels in HeLa cells following 72 h treatment with 10 μM of each compound.

Alpha-synuclein has been implicated in neurodegenerative disorders characterized by Lewy body inclusions such as Parkinson's disease (PD) and dementia with Lewy bodies. Lewy body-like inclusions have also been observed in spinal neurons of patients with amyotrophic lateral sclerosis (ALS) and reports suggest possible alpha-synuclein abnormalities in ALS patients alpha-Synuclein is a ubiquitous protein that shares significant physical and functional homology to the protein chaperone, 14-3-3, and is particularly abundant in the brain (Ostrerova N. et al., J. Neurosci., 19:5782 (1990)). An increased rate of alpha-synuclein aggregation might contribute to the mechanisms of neurodegeneration in Lewy body diseases. Studies on transgenic animals also suggest that aggregation of alpha-synuclein is harmful to neurons. It was reported that dopaminergic dysfunction occurred in transgenic mice expressing wild type human alpha-synuclein (Masliah, E., et at., Science, 287:1265-1269 (2000)) and that Drosophila over-expressing alpha-synuclein exhibited dopaminergic dysfunction and dopaminergic neuronal death associated with development of alpha-synuclein aggregates (Feany, M B, et al., Nature 404:394-8 (2000)). Evidence suggests that neurons with dopamine develop alpha-synuclein aggregates and degenerate as these aggregates develop.

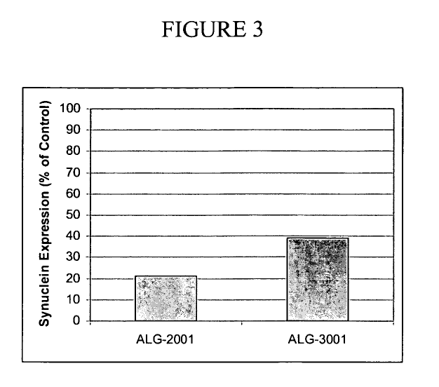

The results of the Genechip analysis shown in FIG. 3 illustrates that pyrimethamine (ALG-2001) (3 μM) and norethindrone (ALG-3001) (3 μM) substantially decreased mRNA for alpha synuclein in HeLa cells following 4 days of treatment. Thus, the compounds of the present invention can slow neurodegeneration in Lewy body diseases. This illustrates a role for the compounds of the present invention in slowing the progression or ameliorating the effects of ALS and PD.

### Example 3

### Testing the Effects of Pharmacological Agents In vivo

(a) SOD-93A Murine Model

The effects of the pharmacological agents e.g., pyrimethamine, and analogs thereof described in Example 2 were tested in vivo in the SOD-93A murine model for ALS and a reduction in the SOD-1 levels was measured. The inhibition of RNA expression was monitored by isolated blood samples from the art recognized mouse model of ALS pre- and post introduction of the compound using standard RT-PCR techniques. The expression of the SOD-1 protein was determined using Western blot techniques with an anti-SOD-1 antibody from Sigma.

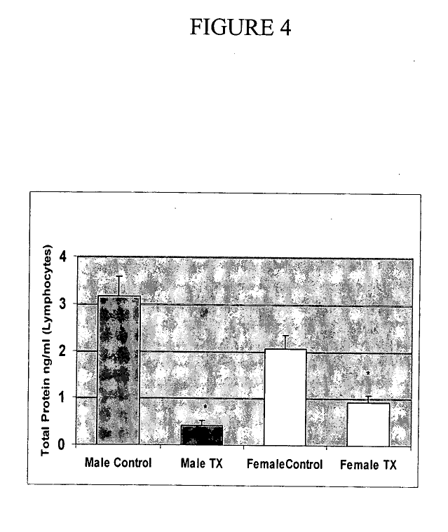

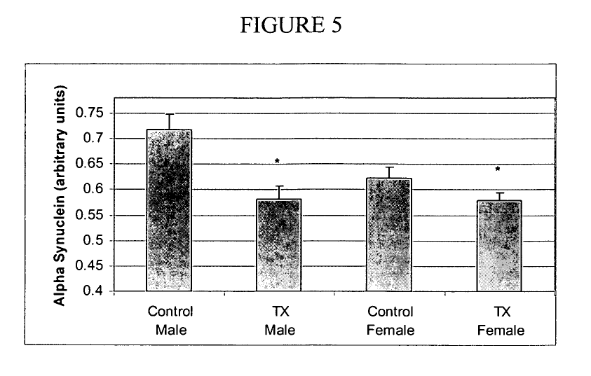

As shown in FIGS. 4 and 5, chronic treatment with pyrimethamine (10 mg/kg ip×14 days) significantly (P<0.05, n=7) decreased SOD-1 protein and alpha synuclein in mouse lymphocytes. The results were show to be statistically significant using a student t-test analysis. The control was vehicle (saline).

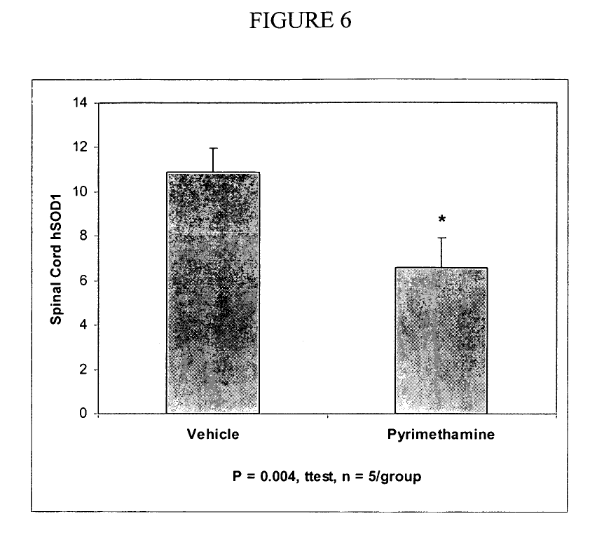

Chronic pyrimethamine (50 mg/kg/d) significantly decreased spinal SOD-1 in G93A mice following 14 d treatment as shown in FIG. 6. Pyrimethamine was administered orally for 14 days. Spinal cords were harvested and analyzed by Western blot analysis as described above.

(b) Human Familial ALS Patient

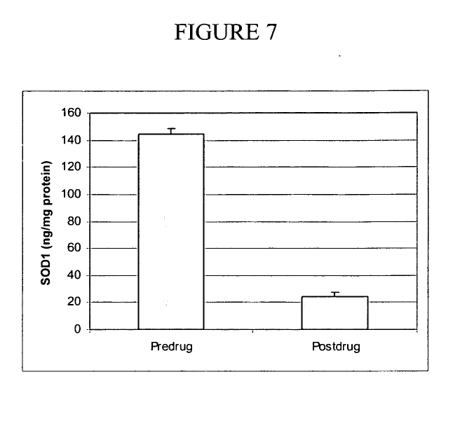

A 38 year old familial ALS patient volunteer showed a significant decrease in SOD-1 levels following oral treatment with pyrimethamine (100 mg/d for 30 days). FIG. 7 shows the decreased lymphocyte SOD1 levels in the familial SOD1 patient following administration of the drug (post drug) compared to prior to treatment (predrug). Approximately 5-8 cc blood was collected from the patient. SOD-1 levels were analyzed by ELISA and Western blot analysis.

The in vivo effects can also be determined by monitoring the breathing of a subject by measuring the forced vital capacity (FVC) using a Renaissance Puritan Bennett Spirometer. The maximum inspiratory force (MIF) can also be measured using a hand held manometer. Motorneuron loss can be monitored by the MUNE techniques (Aggarwal et. al, J. Neurol. Neurosurg. Psychiatry, August 2002; 73: 199-201.). Clinical symptoms including strength can be measured using manual muscle testing (MMT), the Appel Scale, or the ALS functional rating scale (ALSFRS/ALSFRS-R). (Couratier P, Rev Neurol (Paris). 2006; 162(4):502-7)

### Example 4

### Neurological Scoring

The effects of the nuclear receptor modulating pharmacological agents can also be determined by a neurological score recorded on a 4-point scale:

| | |
|---|---|
| 0 = | Normal reflex on the hind limbs (animal will splay its hind |
| | limbs when lifted by its tail) |
| 1 = | Abnormal reflex (Lack of splaying of hind limbs when animal is |
| | lifted by the tail). |
| 2 = | Abnormal reflex and visible evidence of paralysis |
| 3 = | Lack of reflex and total paralysis of hind limbs. |
| 4 = | Inability to right themselves when placed on the sides in 30 |
| | seconds or found dead. The animals are sacrificed at this stage |
| | if alive. |

Statistical analysis on the neurological score, body weight and survival can be performed by utilizing ANOVA, Kaplan Meier, t-test, Cox's proportional hazards regression model, log-logistic and parametric methods and mixed linear model methods. All statistical analysis was performed using standard procedures known in the art.

1. A method for reducing the production of an SOD protein in a cell comprising, administering a pharmacological agent to the cell, such that the agent decreases levels of the SOD protein, wherein the agent is a compound of Formula I 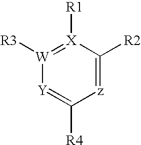
wherein W, X, Y, and Z are independently selected from the group consisting of C, N, O, and S, with at least one being non-carbon,
R1, R2, R3, and R4 are independently selected from the group consisting of hydrogen, halogen, cyano, SCN, alkyl, haloalkyl, hydroxyalkyl, alkoxyalkyl, aryloxyalkyl, carboxyalkyl, carboalkoxyalkyl, arylalkyl, heteroarylalkyl, cycloalkyl, mercaptoalkyl, alkylthioalkyl, alkylsulfonylalkyl, alkylsulfoxylalkyl, acyl (eg. acetyl), alkenyl, alkynyl, arylalkenyl, arylalkynyl, aryloxyalkenyl, aryloxyalkynyl, arylthioalkenyl, arylthioalkynyl, heteroarylalkenyl, heteroarylalkynyl, heteroaryloxyalkenyl, heteroaryloxyalkynyl, heteroarylthioalkenyl, heteroarylthioalkynyl, aryl, heteroaryl, aroyl (eg. benzoyl), heteroaroyl, and saturated heterocyclyl (eg. morpholino, thiamorpholino, piperazinyl, piperidinyl, pyrrolidinyl, tetrahydrofuranyl), nitro, amino, alkylamino, dialkylamino, arylamino, heteroarylamino, arylalkylamino, heteroarylalkylamino, acylamino, aroylamino, heteroaroylamino, arylguanidino, ring fusions, CONRaryl, CONRheteroaryl, CO₂H, CO₂R, hydroxy or double-bonded oxygen, alkoxy, aryloxy, heteroaryloxy, haloalkoxy, carboxyalkoxy, carboalkoxyalkoxy, aryloxy, heteraryloxy, alkenyloxy, alkynyloxy, OCH₂CO₂R, OCH₂CONR₂, mercapto, alkylthio, alkylsulfonyl, alkylsulfoxyl, arylthio, heteroarylthio, arylalkylthio, heteroarylalkylthio, alkenylthio, alkynylthio, SCH₂CO₂R, and SCH₂CONR₂, where R═H, alkyl, haloalkyl, or alkoxyalkyl.

2. The method of claim 1, wherein the cell is selected from the group consisting of a cell in a brain, a cell in a spinal cord, a cell in a meningial tissue and, a cell in muscle.

3. The method of claim 1, wherein the cell is a neural, other central nervous system, or muscle cell in a subject with ALS.

4. The method of claim 1, wherein the SOD protein is the SOD-1 protein.

5. The method of claim 1, wherein the pharmacological agent is a pyrimidine of formula

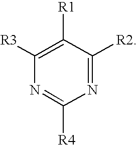

6. The method of claim 5, wherein R1 is a haloaryl.

7. The method of claim 6, wherein R1is 4-C₆H₄—Cl.

8. The method of claim 7 wherein R4 is SH or NH₂.

9. The method of claim 8 wherein R2 is NH₂ and R3 is C₂H₅.

10. The method of claim 1, wherein the pharmacological agent is a 1,3,5-triazine of formula

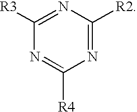

11. The method of claim 10, wherein R2 is a haloalkyl.

12. The method of claim 11, wherein R2 is CHCl₂.

13. The method of claim 12, wherein R3 is an alkylthio.

14. The method of claim 13, wherein R4 is an aryl.

15. The method of claim 1, wherein the pharmacological agent is a quinazoline of formula

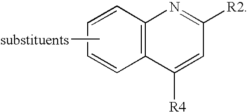

16. The method of claim 15, wherein R4 is a saturated heterocycle.

17. The method of claim 16, wherein R4 is 4-methylpiperazin-1-yl.

18. The method of claim 17, wherein R2 is CF3.

19. The method of claim 1, wherein the pharmacological agent is a heterocyclic amide of Formula I with W,X,Z=C; Y═N and R4=NRCOaryl.

20. The method of claim 1, wherein the pharmacological agent is a flavonoid of Formula I with W,Y,Z=C; X═O and R3 is a benzo fused ring.

21. The method of claim 20, wherein the pharmacological agent is a flavonoid of Formula I with R4=double bonded oxygen.

22. The method of claim 21, wherein the pharmacological agent is a flavonoid of Formula I with R2=H or aryl.

23. A method for preventing the development of symptoms, or ameliorating the symptoms or progression of amyotrophic lateral sclerosis (ALS) in a subject comprising, administering a prophylactically or therapeutically effective amount of a pharmacological agent to the subject, wherein the agent decreases levels of the SOD-1 protein.

24. The method of claim 23, wherein the pharmacological agent is a pyrimidine of Formula I, wherein W and X are C; and Y and Z is N.

25. The method of claim 24 wherein the pharmacological agent is a pyrimidine of Formula I wherein R1 is a haloaryl.

26. The method of claim 25 wherein the pharmacological agent is a pyrimidine of Formula I wherein R1 is 4-C₆H₄—Cl.

27. The method of claim 26 wherein the pharmacological agent is a pyrimidine of Formula I wherein R4 is SH or NH₂.

28. The method of claim 27 wherein the pharmacological agent is the pyrimidine of Formula I wherein R2 is NH₂ and R3 is C₂H₅.

29. The method of claim 23, wherein the pharmacological agent is a 1,3,5-triazine of Formula I, wherein W is C; and X,Y, and Z is N.

30. The method of claim 29, wherein the pharmacological agent is a 1,3,5-triazine of Formula I wherein R2=haloalkyl.

31. The method of claim 30, wherein the pharmacological agent is a 1,3,5-triazine of Formula I wherein R2=CHCl₂.

32. The method of claim 31, wherein the pharmacological agent is a 1,3,5-triazine of Formula I wherein R3 is an alkylthio.

33. The method of claim 32, wherein the pharmacological agent is a 1,3,5-triazine of Formula I wherein R4 is an aryl.

34. The method of claim 23, wherein the pharmacological agent is a quinazoline of Formula I wherein W,Y, and Z is C; X is N; and R3 is a benzo fused ring.

35. The method of claim 34, wherein the pharmacological agent is a quinazoline of Formula I wherein R4 is a saturated heterocycle.

36. The method of claim 35, wherein the pharmacological agent is a quinazoline of Formula I wherein R4 is 4-methylpiperazin-1-yl.

37. The method of claim 36, wherein the pharmacological agent is a quinazoline of Formula I wherein R2 is CF₃.

38. The method of claim 23, wherein the pharmacological agent is a heterocyclic amide of Formula I wherein W, X, and Z is C; Y is N and R4 is NRCOaryl.

39. The method of claim 23, wherein the pharmacological agent is a flavonoid of Formula I wherein W, Y, and Z is C; X is O and R3 is a benzo fused ring.

40. The method of claim 39, wherein the pharmacological agent is a flavonoid of Formula I wherein R4 is a double bonded oxygen, and R2 is H or aryl.

41. The method of claim 23, further comprising monitoring the amelioration of ALS by monitoring survival prolongation of the subject.

42. The method of claim 41, wherein the step of monitoring the amelioration of ALS comprises monitoring a neurological score of the subject.

43. The method of claim 41, wherein the step of monitoring the amelioration of ALS comprises monitoring expression levels of the SOD-1 protein.

44. The method of claim 41, wherein the step of monitoring the amerlioration of ALS comprises monitoring the clinical measures MMT, ALSFRS/ALSFRS-R, or the Appel Scale.

45. The method of claim 41, wherein the step of monitoring the amerlioration of ALS comprises monitoring the number of motor units via the MUNE technique.
